SEQUENCE LISTING (5 sequences)

SEQ ID NO: 1 - protein, 16 aa, Artificial Sequence
  Description of Artificial Sequence Synthetic peptide
RDHMVLHEYVNAAGIT

SEQ ID NO: 2 - protein, 320 aa, Homo sapiens
MSKSASPKEPEQLRKLFIGGLSFETTDESLRSHFEQWGTLTDCVVMRDPNTKRSRGFGFVTYATVEEVDAAMNTTPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVKGHNCEVRKALPKQEMASASSSQRGRRGSGNFGGGRGDGFGGNDNFGRGGNFSGRGGFGGSCGGGGYGGSGDGYNGFGNDGSNFGGGGSYNDFGNYNNQSSNFGPMKGGNFGGRSSGPYGGGGQYFAKPQNQGGYGVSSSSSSYGSGRRF

SEQ ID NO: 3 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 4 - protein, 724 aa, Homo sapiens
MGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVTYIPPPPPEDEDSIFAHYQTGINFDKYDTILVEVSGHDAPPAILTFEEANLCQTLNNNIAKAGYTKLTPVQKYSIPIILAGRDLMACAQTGSGKTAAFLLPILAHMMHDGITASRFKELQEPECIIVAPTRELVNQIYLEARKFSFGTCVRAVVIYGGTQLGHSIRQIVQGCNILCATPGRLMDIIGKEKIGLKQIKYLVLDEADRMLDMGFGPEMKKLISCPGMPSKEQRQTLMFSATFPEEIQRLAAEFLKSNYLFVAVGQVGGACRDVQQTVLQVGQFSKREKLVEILRNIGDERTMVFVETKKKADFIATFLCQEKISTTSIHGDREQREREQALGDFRFGKCPVLVATSVAARGLDIENVQHVINFDLPSTIDEYVHRIGRTGRCGNTGRAISFFDLESDNHLAQPLVKVLTDAQQDVPAWLEEIAFSTYIPGFSGSTRGNVFASVDTRKGKSTLNTAGFSSSQAPNPVDDESWD

SEQ ID NO: 5 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

(19) United States
(12) Patent Application Publication
(10) Pub. No.: US 2024/0302350 A1
(43) Pub. Date: Sep. 12, 2024

(54) METHODS OF SCREENING INHIBITORS OF BIOMOLECULAR INTERACTIONS USING PHASE SEPARATION AS IN CELLULO READ-OUT

(71) Applicant: The Trustees of Princeton University, Princeton, NJ (US)

(72) Inventors: Clifford P. BRANGWYNNE, Hopewell, NJ (US); Chanelle JUMPER, Princeton, NJ (US); Paul ACKERMAN, Princeton, NJ (US); Dan BRACHA, Princeton, NJ (US)

(73) Assignee: The Trustees of Princeton University, Princeton, NJ (US)

(21) Appl. No.: 18/273,816

(22) PCT Filed: Jan. 21, 2022
(86) PCT No.: PCT/US2022/013213

§ 371 Date: Feb. 6, 2024

Related U.S. Application Data
(60) Provisional application No. 63/140,291, filed on Jan. 22, 2021.

(51) Int. Cl. G01N 33/50; G01N 33/68

(52) U.S. Cl. CPC G01N 33/5038; G01N 33/6845

(57) ABSTRACT

Provided herein are methods of identifying an inhibitory activity of an exogeneous agent that disrupts binding of a target peptide to a biomolecule using phase separation as in cellulo read-out. Provided herein also are kits for the methods, pharmaceutical compositions comprising the agents screened by the methods, methods of preparing the pharmaceutical composition, and methods of treating a disease or condition in a subject in need thereof by administering the pharmaceutical composition.

### CROSS-REFERENCE

This application claims the benefit of U.S. Provisional Application No. U.S. 63/140,291, filed Jan. 22, 2021, which is incorporated herein by reference in its entirety.

### REFERENCE TO SEQUENCE LISTING SUBMITTED ELECTRONICALLY

The Sequence Listings filed electronically herewith is also hereby incorporated by reference in its entirety (File Name: Princeton-73476_SL.txt, Date Created: Jan. 19, 2022; File Size: 17,378 bytes).

### BACKGROUND

Assays for the identification of inhibitors for disease-associated biomolecular interactions are most often performed in vitro and rely on extraction and/or purification of components combined with lengthy assay development and validation. In order to perform in cellulo screening, which is desirable for targeting interactions in their native environments and in the presence of their complete biochemical pathways, assays typically require years of development and validation. Thus, a technique for overcoming that requirement is in great need.

### SUMMARY

Described herein, in certain embodiments, is a method of identifying an inhibitory activity of an exogeneous agent that disrupts binding of a target peptide to a biomolecule, the method comprising: (i) contacting a cell with the agent, wherein the cell comprises a target molecule that comprises the target peptide; (ii) detecting membrane-less bodies comprising the target molecule, wherein the target molecule is incorporated into the membrane-less bodies when the target peptide is released from the binding to the biomolecule and undergoes phase separation at a sub-region of the cell that is separate and distinct from a sub-region where the target peptide binds to the biomolecule; wherein the detecting comprises measuring a change in an intensity and a size of the membrane-less bodies; and wherein a bigger change in the intensity and the size of the membrane-less bodies relative to a change in an intensity and a size of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a first cell with the agent, wherein the first cell comprises a target molecule that comprises the target peptide; (ii) contacting a second cell with the agent, wherein the second cell comprises a control molecule that comprises a control peptide, wherein the control peptide is a non-target peptide; (iii) detecting first membrane-less bodies of the first cell comprising the target molecule, wherein the target molecule is incorporated into the first membrane-less bodies when the target peptide is released from the binding to the biomolecule and undergoes phase separation at a sub-region of the cell that is separate and distinct from a sub-region where the target peptide binds to the biomolecule, and wherein the detecting comprises measuring a change in an intensity and a size of the first membrane-less bodies, and detecting second membrane-less bodies of the second cell comprising the control molecule, wherein the control molecule is incorporated into the second membrane-less bodies when the control peptide undergoes phase separation, and wherein the detecting comprises measuring a change in an intensity and a size of the second membrane-less bodies; and (iv) determining a difference between the change in the intensity and the size of the first membrane-less bodies and the change in the intensity and the size of the second membrane-less bodies; wherein a bigger difference between the first membrane-less bodies and the second membrane-less bodies relative to a difference between the first membrane-less bodies and the second membrane-less bodies in the absence of the agent indicates that the agent disrupts binding of the target peptide to the biomolecule.

In some embodiments, the target peptide is fused to a fluorophore. In some embodiments, the target peptide is fused to a first fluorophore and the control peptide is fused to a second fluorophore. In some embodiments, the detecting membrane-less bodies is measuring fluorescence. In some embodiments, the measuring fluorescence comprises detecting an intensity of fluorescence, quantifying the number of the membrane-less bodies, or a combination thereof.

In some embodiments, the target peptide is an intrinsically disordered protein (IDP) or the target peptide comprises an intrinsically disordered region (IDR). In some embodiments, the membrane-less bodies, first membrane-less bodies, or second membrane-less bodies are nucleoli, Cajal bodies, Stress granule, P-bodies, spliceosomes, or a combination thereof.

In some embodiments, the biomolecule is a protein, a protein domain, DNA, RNA, or a complex comprising a protein, DNA, RNA, or a combination thereof.

In some embodiments, the agent is a small molecule compound. In some embodiments, the small molecule compound is an organic compound. In some embodiments, the small molecule compound is an inorganic compound. In some embodiments, the agent is selected from the group consisting of a nucleic acid, a polynucleotide, a polypeptide, or a combination thereof. In some embodiments, the polypeptide is an antibody or a functional fragment thereof. In some embodiments, the polynucleotide is an aptamer, a RNA-based compound, an antisense nucleic acid, a PNA, or an combination thereof. In some embodiments, the RNA-based compound is a small interfering RNA, a microRNA, a small hairpin RNA, or a combination thereof. In some embodiments, the polynucleotide is a CRISPR guide RNA. In some embodiments, the CRISPR guide RNA is encoded by a viral vector. In some embodiments, the viral vector is a lentiviral vector. In some embodiments, the CRISPR guide RNA is introduced in combination with Cas9 or a vector comprising a nucleotide sequence encoding the Cas9. In some embodiments, the Cas9 is enzymatically dead Cas9 (dCas9).

In some embodiments, the target molecule is a target protein.

In some embodiments, the target molecule further comprises a full length or truncated low complexity region fused to the target peptide. In some embodiments, the control molecule further comprises the full length or truncated low complexity region fused to the control peptide. In some embodiments, the target molecule further comprises an additional full length or truncated IDR fused to the target peptide, wherein the additional full length or truncated IDR is different from the target molecule. In some embodiments, the control molecule further comprises the additional full length or truncated IDR fused to the control peptide. In some embodiments, the IDR is at least a portion of FUS, a portion of Ddx4, a portion of hnRNPA1, a portion of BRD4, a portion of TAF15, a portion of SRSF2 IDR, a portion of SART1, a portion of HSF1, a portion of RNPS1, or a combination thereof. In some embodiments, the portion of FUS is an N-terminal IDR of FUS (FUSn).

In some embodiments, the fluorophore is mCherry or green fluorescent protein (GFP).

Described herein, in certain embodiments, is a method of identifying an inhibitory activity of an exogeneous agent that disrupts binding of a target peptide to a biomolecule, the method comprising: (i) contacting a cell with the agent, wherein the cell comprises a target molecule that comprises the target peptide fused to a fluorophore; (ii) measuring fluorescence of membrane-less bodies comprising the target molecule by detecting an intensity of fluorescence, quantifying the number of the membrane-less bodies, or a combination thereof, wherein the target molecule is incorporated into the membrane-less bodies when the target peptide is released from the binding to the biomolecule and undergoes phase separation at a sub-region of the cell that is separate and distinct from a sub-region where the target peptide binds to the biomolecule; wherein the fluorophore is mCherry and the measuring fluorescence is performed by exposing the cell to 540-590 nm wavelength of light and imaging the cell at 550-650 nm wavelength of light, or wherein the fluorophore is GFP and the measuring fluorescence is performed by exposing the cell to 488 nm wavelength of light and imaging the cell at 510 nm wavelength of light; and wherein a higher level of the fluorescence of the membrane-less bodies relative to a level of fluorescence of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a cell with the agent, wherein the cell comprises a target molecule that comprises the target peptide, and at least one peptide selected from a light-sensitive receptor and a chemical-sensitive receptor; (ii) exposing the cell to at least one predetermined wavelength of light, a chemical to which the chemical-sensitive receptor is sensitive, or a combination thereof; and (iii) detecting membrane-less bodies comprising the target molecule, wherein the target molecule is incorporated into the membrane-less bodies when the target peptide is released from the binding to the biomolecule and undergoes phase separation at a sub-region of the cell that is separate and distinct from a sub-region where the target peptide binds to the biomolecule; wherein the detecting comprises measuring a change in an intensity and a size of the membrane-less bodies; and wherein a bigger change in the intensity and the size of the membrane-less bodies relative to a change in an intensity and a size of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a cell with the agent, wherein the cell comprises (a) a target molecule that comprises the target peptide and a peptide selected from a group consisting of a light-sensitive receptor, a chemical-sensitive receptor, a light-sensitive oligomerization protein, and a non-light sensitive dimerization module; and (b) a second molecule comprising a cognate partner of the peptide fused to a full length or truncated low complexity protein region or a full length or truncated intrinsically-disordered protein region (IDR); (ii) exposing the cell to at least one predetermined wavelength of light, a chemical to which the chemical-sensitive receptor is sensitive, or a combination thereof; and (iii) detecting membrane-less bodies comprising the target molecule, wherein the target molecule and the second molecule are incorporated into the membrane-less bodies when the target peptide is released from the binding to the biomolecule, the peptide of the target molecule interacts with the cognate partner of the second molecule, and the target molecule and the second molecule undergo phase separation at a sub-region of the cell that is separate and distinct from a sub-region where the target peptide binds to the biomolecule, wherein the detecting comprises measuring a change in an intensity and a size of the membrane-less bodies; and wherein a bigger change in the intensity and the size of the membrane-less bodies relative to a change in an intensity and a size of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a cell with the agent, wherein the cell comprises (a) a target molecule that comprises the target peptide and a cognate partner of a peptide selected from a group consisting of a light-sensitive receptor, a chemical-sensitive receptor, a light-sensitive oligomerization protein, and a non-light sensitive dimerization module; and (b) a second molecule comprising the peptide fused to a full length or truncated low complexity protein region or a full length or truncated intrinsically-disordered protein region (IDR); (ii) exposing the cell to at least one predetermined wavelength of light, a chemical to which the chemical-sensitive receptor is sensitive, or a combination thereof; and (iii) detecting membrane-less bodies comprising the target molecule, wherein the target molecule and the second molecule are incorporated into the membrane-less bodies when the target peptide is released from the binding to the biomolecule, the cognate partner of the target molecule interacts with the peptide of the second molecule, and the target molecule and the second molecule undergo phase separation at a sub-region of the cell that is separate and distinct from a sub-region where the target peptide binds to the biomolecule, wherein the detecting comprises measuring a change in an intensity and a size of the membrane-less bodies; and wherein a bigger change in the intensity and the size of the membrane-less bodies relative to a change in an intensity and a size of membrane-less bodies in the absence of the agent indicates that the agent disrupts binding of the target peptide to the biomolecule.

In some embodiments, the target molecule further comprises a fluorophore fused between the target peptide and the at least one peptide selected from a light-sensitive receptor and a chemical-sensitive receptor. In some embodiments, the target molecule further comprises a fluorophore fused between the target peptide and the peptide selected from a group consisting of a light-sensitive receptor, a chemical-sensitive receptor, a light-sensitive oligomerization protein, and a non-light sensitive dimerization module. In some embodiments, the target molecule further comprises a fluorophore fused between the target peptide and the cognate partner of the peptide selected from a group consisting of a light-sensitive receptor, a chemical-sensitive receptor, a light-sensitive oligomerization protein, and a non-light sensitive dimerization module. In some embodiments, the detecting membrane-less bodies is measuring fluorescence. In some embodiments, the measuring fluorescence comprises detecting an intensity of fluorescence, quantifying the number of the membrane-less bodies, or a combination thereof. In some embodiments, the membrane-less bodies are nucleoli, Cajal bodies, Stress granule, P-bodies, spliceosomes, or a combination thereof.

In some embodiments, the biomolecule is a protein, a protein domain, DNA, RNA, or a complex comprising a protein, DNA, RNA, or a combination thereof.

In some embodiments, the agent is a small molecule compound. In some embodiments, the small molecule compound is an organic compound or an inorganic compound. In some embodiments, the agent is selected from the group consisting of a nucleic acid, a polynucleotide, a polypeptide, or a combination thereof. In some embodiments, the polypeptide is an antibody or a functional fragment thereof. In some embodiments, the polynucleotide is an aptamer, a RNA-based compound, an antisense nucleic acid, a PNA, or an combination thereof. In some embodiments, the RNA-based compound is a small interfering RNA, a microRNA, a small hairpin RNA, or a combination thereof. In some embodiments, the polynucleotide is a CRISPR guide RNA. In some embodiments, the CRISPR guide RNA is encoded by a viral vector. In some embodiments, the viral vector is a lentiviral vector. In some embodiments, the CRISPR guide RNA is introduced in combination with Cas9 or a vector comprising a nucleotide sequence encoding the Cas9. In some embodiments, the Cas9 is enzymatically dead Cas9 (dCas9).

In some embodiments, the IDR is at least a portion of FUS, a portion of Ddx4, a portion of hnRNPA1, a portion of BRD4, a portion of TAF15, a portion of SRSF2 IDR, a portion of SART1, a portion of HSF1, a portion of RNPS1, or a combination thereof. In some embodiments, the portion of FUS is an N-terminal IDR of FUS (FUSn).

In some embodiments, the light-sensitive receptor is cryptochrome 2 (Cry2), a photolyase homology region of Cry2, phytochrome B (PhyB), PIF, a light oxygen voltage sensing (LOV) domain, Dronpa, or an improved light-induced dimer (iLID). In some embodiments, the light-sensitive receptor is ssrA and the cognate partner is sspB.

In some embodiments, the fluorophore is mCherry or green fluorescent protein (GFP).

In some embodiments, the cell is a human cell. In some embodiments, the cell is a non-human animal cell.

In some embodiments, the target molecule (e.g., target protein) is BRD4 or a bromodomain of BRD4. In some embodiments, the biomolecule is an acetylated histone.

In some embodiments, the target molecule (e.g., target protein) is a transcriptional activator or a transcriptional repressor. In some embodiments, the biomolecule is DNA. In some embodiments, the biomolecule is a basic helix-loop-helix domain of DNA.

In some embodiments, the agent is a therapeutic agent.

Described herein, in certain embodiments, is a method of determining effectiveness of a treatment regimen in a subject, comprising: (i) treating the subject with an agent that disrupts binding of a target peptide to a biomolecule; (ii) forming membrane-less bodies in a cell from the subject, the cell comprising a target molecule that comprises the target peptide; wherein the target molecule is incorporated into the membrane-less bodies when the target peptide is released from the biomolecule and undergoes phase separation at a sub-region of the cell that is separate from a sub-region where the target peptide binds to the biomolecule; and (iii) determining effectiveness of the treatment regimen by detecting a change in an intensity or a size of the membrane-less bodies as compared to an intensity or size of membrane-less bodies in the absence of the agent.

Described herein, in certain embodiments, is a method of modulating interaction between a target peptide and a biomolecule in a cell of a subject, the cell comprising a target molecule that comprises the target peptide, the method comprising: (i) contacting the cell with an agent that disrupts binding of the target peptide to the biomolecule so as to release the target peptide from the biomolecule; (ii) incorporating the target molecule into membrane-less bodies when the target peptide is released from the biomolecule, wherein the target molecule undergoes phase separation at a sub-region of the cell that is separate from a sub-region where the target peptide binds to the biomolecule; thereby modulating the target peptide and the biomolecule.

Described herein, in certain embodiments, is a method of modulating subcellular localization of a target molecule in a cell of a subject, comprising: (i) contacting the cell with an agent that disrupts binding of a target peptide to a biomolecule so as to release the target peptide from the biomolecule; (ii) incorporating the target molecule into membrane-less bodies when the target peptide is released from the biomolecule, wherein the target molecule undergoes phase separation at a sub-region of the cell that is separate from a sub-region where the target peptide binds to the biomolecule; thereby modulating interaction between the target peptide and the biomolecule.

In some embodiments, the method further comprises detecting the membrane-less bodies in the cell, wherein the membrane-less bodies comprise the target molecule.

In some embodiments, the target peptide is fused to a fluorophore. In some embodiments, the detecting membrane-less bodies is measuring fluorescence. In some embodiments, the measuring fluorescence comprises detecting an intensity of fluorescence, quantifying the number of the membrane-less bodies, or a combination thereof.

Described herein, in certain embodiments, is a method of identifying an inhibitory activity of an exogeneous agent that disrupts binding of a target peptide to a biomolecule, the method comprising: (i) contacting a cell with the agent, wherein the cell comprises a target molecule that comprises the target peptide, at least one light-sensitive receptor, and a fluorophore fused between the target peptide and the at least one light-sensitive receptor; (ii) exposing the cell to at least one predetermined wavelength of light; and (iii) measuring fluorescence of membrane-less bodies comprising the target molecule by detecting an intensity of fluorescence, quantifying the number of the membrane-less bodies, or a combination thereof, wherein the target molecule is incorporated into the membrane-less bodies when the target peptide is released from the binding to the biomolecule and undergoes phase separation at a sub-region of the cell that is separate and distinct from a sub-region where the target peptide binds to the biomolecule; wherein the at least one wavelength of light is a visible wavelength of light between and including 400 and 800 nm; wherein the fluorophore is mCherry and the measuring fluorescence is performed by exposing the cell to 540-590 nm wavelength of light and imaging the cell at 550-650 nm wavelength of light, or wherein the fluorophore is GFP and the measuring fluorescence is performed by exposing the cell to 488 nm wavelength of light and imaging the cell at 510 nm wavelength of light; and wherein a higher level of the fluorescence of the membrane-less bodies relative to a level of fluorescence of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a cell with the agent, wherein the cell comprises (a) a target molecule that comprises the target peptide, a light-sensitive receptor, and a fluorophore fused between the target peptide and the light-sensitive receptor; and (b) a second molecule comprising a cognate partner of the light-sensitive receptor fused to a full length or truncated low complexity protein region or a full length or truncated intrinsically-disordered protein region (IDR); (ii) exposing the cell to at least one predetermined wavelength of light; and (iii) measuring fluorescence of membrane-less bodies comprising the target molecule by detecting an intensity of fluorescence, quantifying the number of the membrane-less bodies, or a combination thereof, wherein the target molecule and the second molecule are incorporated into the membrane-less bodies when the target peptide is released from the binding to the biomolecule, the peptide of the target molecule interacts with the cognate partner of the second molecule, and the target molecule and the second molecule undergo phase separation at a sub-region of the cell that is separate and distinct from a sub-region where the target peptide binds to the biomolecule; wherein the light-sensitive receptor is Cry2 or iLID, and the at least one predetermined wavelength of light is 450 nm wavelength of light or 440 nm wavelength of light in junction with 488 nm wavelength of light, or wherein the light-sensitive receptor is PhyB or PIF, and the at least one predetermined wavelength of light is 650 nm wavelength of light in junction with 750 nm wavelength of light; wherein the fluorophore is mCherry and the measuring fluorescence is performed by exposing the cell to 540-590 nm wavelength of light and imaging the cell at 550-650 nm wavelength of light, or wherein the fluorophore is GFP and the measuring fluorescence is performed by exposing the cell to 488 nm wavelength of light and imaging the cell at 510 nm wavelength of light; and wherein a higher level of the fluorescence of the membrane-less bodies relative to a level of fluorescence of membrane-less bodies in the absence of the agent indicates that the agent disrupts binding of the target peptide to the biomolecule.

Described herein, in certain embodiments, is a method of identifying an inhibitory activity of an exogeneous agent that disrupts binding of a target peptide to a biomolecule, the method comprising: (i) contacting a cell with the agent, wherein the cell comprises (a) a target molecule that comprises the target peptide, a cognate partner of a light-sensitive receptor, and a fluorophore fused between the target peptide and the cognate partner; and (b) a second molecule comprising the light-sensitive receptor fused to a full length or truncated low complexity protein region or a full length or truncated intrinsically-disordered protein region (IDR); (ii) exposing the cell to at least one predetermined wavelength of light; and (iii) measuring fluorescence of membrane-less bodies comprising the target molecule by detecting an intensity of fluorescence, quantifying the number of the membrane-less bodies, or a combination thereof, wherein the target molecule and the second molecule are incorporated into the membrane-less bodies when the target peptide is released from the binding to the biomolecule, the peptide of the target molecule interacts with the cognate partner of the second molecule, and the target molecule and the second molecule undergo phase separation at a sub-region of the cell that is separate and distinct from a sub-region where the target peptide binds to the biomolecule; wherein the light-sensitive receptor is Cry2 or iLID, and the at least one predetermined wavelength of light is 450 nm wavelength of light or 440 nm wavelength of light in junction with 488 nm wavelength of light, or wherein the light-sensitive receptor is PhyB or PIF, and the at least one predetermined wavelength of light is 650 nm wavelength of light in junction with 750 nm wavelength of light; wherein the fluorophore is mCherry and the measuring fluorescence is performed by exposing the cell to 540-590 nm wavelength of light and imaging the cell at 550-650 nm wavelength of light, or wherein the fluorophore is GFP and the measuring fluorescence is performed by exposing the cell to 488 nm wavelength of light and imaging the cell at 510 nm wavelength of light; and wherein a higher level of the fluorescence of the membrane-less bodies relative to a level of fluorescence of membrane-less bodies in the absence of the agent indicates that the agent disrupts binding of the target peptide to the biomolecule. In certain embodiments, the GFP is GFP11 (SEQ ID NO: 1).

Described herein, in certain embodiments, is a pharmaceutical composition comprising the agent as described herein, and a pharmaceutically acceptable excipient, diluent, or carrier.

Described herein, in certain embodiments, is a method of preparing the pharmaceutical composition as described herein.

Described herein, in certain embodiments, is a method of treating a disease or condition in a subject in need thereof, comprising administering the pharmaceutical composition as described herein, wherein the administering reduces a sign or a symptom associated with the disease or condition, thereby treating the disease or condition in the subject. In some embodiments, the disease or condition is cancer, a neurodegenerative disorder, or an inflammatory disorder. In some embodiments, the subject is human.

Described herein, in certain embodiments, is a kit, wherein the kit comprises the cell or the first cell and the second cell as described herein; or the target molecule, the target molecule and the control molecule, or the target molecule and the second molecule as described herein; and instructions for the methods as described herein.

### BRIEF DESCRIPTION OF THE DRAWINGS

FIGS. 1A-1E depict exemplary embodiments of the target molecules used for the methods of identifying an inhibitory activity of an exogeneous agent that disrupts binding of a target peptide to a biomolecule as described herein. FIG. 1A depicts an exemplary embodiments of a target molecule that comprises a target peptide fused to a fluorophore. FIG. 1B depicts an exemplary embodiments of a target molecule that comprises a target peptide fused to a fluorophore, and a full length or truncated low complexity region or a full length or truncated intrinsically disordered region (IDR) fused to the target peptide. FIG. 1C depicts an exemplary embodiments of a target molecule that comprises a target peptide, at least one light-sensitive receptor, and a fluorophore fused between the target peptide and the one light-sensitive receptor. FIG. 1D depicts an exemplary embodiments of a target molecule that comprises a target peptide, a light-sensitive receptor, and a fluorophore fused between the target peptide and the light-sensitive receptor; and a second molecule comprising a cognate partner of the light-sensitive receptor fused to a full length or truncated low complexity protein region or a full length or truncated intrinsically-disordered protein region (IDR). FIG. 1E depicts an exemplary embodiments of a target molecule that comprises a target peptide, a cognate partner of a light-sensitive receptor, and a fluorophore fused between the target peptide and the cognate partner; and a second molecule comprising a light-sensitive receptor fused to a full length or truncated low complexity protein region or a full length or truncated intrinsically-disordered protein region (IDR).

FIGS. 2A-2C depict an exemplary embodiment of the methods of identifying an inhibitory activity of an exogeneous agent that disrupts binding of a target peptide to a biomolecule as described herein.

FIG. 2A shows a scheme of an exemplary target molecule that comprises a target peptide (e.g., BRD4), at least one light-sensitive receptor (e.g., sspB), and a fluorophore (mCherry) fused between the target peptide and the one light-sensitive receptor. FIG. 2B depicts images showing that after 90 min 1 μM+JQ1 treatment, the target molecule was released and phase separation was induced compared with untreated cells, and cells at pre-activation.

FIGS. 3A-3D show an exemplary blind screen using the methods of identifying an inhibitory activity of an exogeneous agent that disrupts binding of a target peptide to a biomolecule as described herein. FIG. 3A shows a set-up and coordinates of a multi-well plate. In the “test wells” section of the plate, wells denoted with an X (well ID D04, D06, and D09) are those containing 90 min 1 μM+JQ1 treatment, whereas the blank wells are untreated. Negative control wells are untreated. Gradient wells contain a range of +JQ1 concentrations from 0 to 1 μM. FIG. 3B is the images showing that the cells treated with the candidate agents exhibit the increase of phase separation. FIG. 3C is the graph showing the increase of phase separated cells in the cells treated with some candidate agents (e.g., candidate agents, e.g., +JQ1, in the wells with well ID D04, D06, and D09) as measured by the % of phase separated cells in the population (well). FIG. 3D is the measurement of IC50 for an exemplary agent, 1 μM+JQ1, using the methods as described herein.

FIGS. 4A-4C show an example of the fully sampled IC50 for an exemplary agent, 1 μM+JQ1, using the methods as described herein. FIG. 4A shows a set-up and coordinates of a multi-well plate. FIG. 4B show the serial dilution of +JQ1. FIG. 4C is the graph showing the exemplary results indicating the activity fraction (fraction of phase separating cells) vs concentration of 1 μM+JQ1.

### DETAILED DESCRIPTION

### Definitions

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the use of the singular includes the plural unless specifically stated otherwise. It must be noted that, as used in the specification, the singular forms “a,” “an” and “the” include plural referents unless the context clearly dictates otherwise.

It should also be noted that the term “or” is generally employed in its sense including “and/or” unless the content clearly dictates otherwise.

In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

A “control” or “standard control” refers to a sample, measurement, or value that serves as a reference, usually a known reference, for comparison to a test sample, measurement, or value. A standard control can represent an average measurement or value gathered from a population of similar individuals (e.g., standard control cells or subjects) that, for example, are not treated with the agents tested. A standard control value can also be obtained from the same individual, e.g., from an earlier-obtained sample prior to treatment. Controls are also valuable for determining the significance of data. For example, if values for a given parameter are widely variant in controls, variation in test samples will not be considered as significant. One of skill will recognize that standard controls can be designed for assessment of any number of parameters (e.g., RNA levels, protein levels, specific cell types, specific bodily fluids, specific tissues, brain tissue, etc).

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The terms “polypeptide,” “peptide” and “protein” are used interchangeably in its broadest sense herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid as well as to naturally occurring amino acid polymers and non-naturally occurring amino acid polymers. The subunits may be linked by peptide bonds. In another embodiment, the subunit may be linked by other bonds, e.g., ester, ether, etc.

The term “nucleic acid” refers to deoxyribonucleotides or ribonucleotides and polymers thereof in either single-, double- or multiple-stranded form, or complements thereof. The term “polynucleic acid,” “nucleotide,” or “polynucleotide,” as used herein, refers to a linear sequence of nucleotides. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “transfection,” “transduction,” “transfecting,” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The term “transfection” or “transduction” also refers to introducing proteins into a cell from the external environment. Typically, transduction or transfection of a protein relies on attachment of a peptide or protein capable of crossing the cell membrane to the protein of interest.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The term “fused,” “fusion,” “conjugated,” or “conjugation,” as used herein, refers to a chemical bond that involves the sharing of electron pairs between atoms. In some embodiments, a target peptide is fused or conjugated to a fluorophore. In some embodiments, a fluorophore is fused or conjugated to a target peptide and the at least one peptide selected from a light-sensitive receptor and a chemical-sensitive receptor. In some embodiments, a fluorophore is fused or conjugated to a target peptide and a peptide selected from a group consisting of a light-sensitive receptor, a chemical-sensitive receptor, a light-sensitive oligomerization protein, and a non-light sensitive dimerization module. In some embodiments, a fluorophore is fused or conjugated to a target peptide and a cognate partner of a peptide selected from a group consisting of a light-sensitive receptor, a chemical-sensitive receptor, a light-sensitive oligomerization protein, and a non-light sensitive dimerization module.

The term “binding,” “bind,” “interacting,” “interact,” or “interaction” as used herein, refers to dispersed variations of electromagnetic interactions between molecules or within a molecule, which does not involve the sharing of electrons. Exemplary bindings or interactions include, but are not limited to, electrostatic interactions such as ionic interactions, a hydrogen bonding and a halogen bonding, van der Waals forces such as dipole-dipole interactions, dipole-induced dipole interactions and London dispersion forces, π-effects such as π-π interactions, cation-π and anion-π interactions and polar-π interactions, and hydrophobic effects. In some embodiments, a target molecule binds to or interacts with a biomolecule.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The term “antibody fragment” or “antibody binding domain,” as used herein, refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies such as sdAb (either VL or VH), camelid VHH domains, diabodies, and multi-specific antibodies formed from antibody fragments.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

### Method of Screening Candidate Agents

Described herein, in some embodiments, is a method of identifying an inhibitory activity of an exogeneous agent that disrupts binding of a target peptide to a biomolecule by detecting membrane-less bodies comprising a target molecule. In some embodiments, the target molecule is incorporated into the membrane-less bodies when the target peptide is released from the binding to its biomolecule partner by the inhibitory activity of the agent, and undergoes phase separation at a sub-region of the cell that is separate and distinct from a sub-region where the target peptide binds to the biomolecule. In some embodiments, detecting membrane-less bodies comprises measuring a change in an intensity and a size of the membrane-less bodies. In some embodiments, a change in an intensity of the membrane-less bodies relative to a change in an intensity of membrane-less bodies in the absence of the agent indicates that the agent disrupts binding of the target peptide to the biomolecule. In some embodiments, a change in a size of the membrane-less bodies relative to a change in a size of membrane-less bodies in the absence of the agent indicates that the agent disrupts binding of the target peptide to the biomolecule. In some embodiments, a change in an intensity and a size of the membrane-less bodies relative to a change in an intensity and a size of membrane-less bodies in the absence of the agent indicates that the agent disrupts binding of the target peptide to the biomolecule.

In some embodiments, the change in the intensity of the membrane-less bodies in the presence of an agent is bigger by at least 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, 20000%, 30000%, 40000%, 50000%, 60000%, 70000%, 80000%, 90000%, or 100000% as compared to the change in the intensity of the membrane-less bodies in the absence of the agent.

In some embodiments, the change in the size of the membrane-less bodies in the presence of an agent is bigger by at least 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, 20000%, 30000%, 40000%, 50000%, 60000%, 70000%, 80000%, 90000%, or 100000% as compared to the change in the size of the membrane-less bodies in the absence of the agent.

In some embodiments, the change in the intensity and the size of the membrane-less bodies in the presence of an agent is bigger by at least 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, 20000%, 30000%, 40000%, 50000%, 60000%, 70000%, 80000%, 90000%, or 100000% as compared to the change in the intensity and the size of the membrane-less bodies in the absence of the agent.

In some embodiments, the target molecule comprises a fluorophore and detecting the membrane-less bodies is measuring fluorescence. In some embodiments, the measuring fluorescence comprises detecting an intensity of fluorescence, quantifying the number of the membrane-less bodies, or a combination thereof. In some embodiments, the intensity of fluorescence of the membrane-less bodies in the presence of an agent is higher by at least 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, 20000%, 30000%, 40000%, 50000%, 60000%, 70000%, 80000%, 90000%, or 100000% as compared to an intensity of fluorescence of the membrane-less bodies in the absence of the agent.

In some embodiments, detecting membrane-less bodies comprises the following steps: (i) detecting the intensity and the spatial distribution of fluorescence; (ii) segmenting regions of interest; (iii) a bimodal histogram of intensity for large and optically resolved bodies or large spatial variance of intensities for the case of sub-resolution bodies indicates phase separation. In some embodiments, the existence of phase separation is used as a readout for the presence of membrane-less bodies.

### Liquid-Liquid Phase Separation and Membrane-Less Bodies

Liquid-liquid phase separation (LLPS) is a fundamental mechanism for organizing the contents of living cells. LLPS is now recognized as important for driving assembly of a wide range of membrane-less bodies (membrane-less biomolecular condensates or membrane-less compartments), including cytoplasmic structures such as germ (P) granules, stress granules, miRISC assemblies, and synaptic scaffolds. LLPS also appears to underlie nuclear body biogenesis, including nucleoli, and likely many others. Associated liquid-to-solid phase transitions are also implicated in various diseases of pathological protein aggregation, such as nucleolus-associated diseases, including cancer, ribosomopathies and neurodegeneration, and ageing. Membrane-less droplets inside cells play dynamic roles, for example, in gene regulation and in a host of disease processes. Phase transitions within the nucleus are interesting, since nuclear condensates must directly interact with chromatin, and thus potentially control its organization and gene expression. Consistent with this, the assembly and dynamics of nuclear condensates such as Cajal bodies, nucleoli, and speckles appear to impact chromatin architecture. Phase separation has also been recently implicated in driving gene activation through nanoscale transcriptional condensates assembled at enhancer rich gene clusters.

Intracellular phase transitions arise from weak, multivalent interactions, often mediated by intrinsically disordered proteins/regions (IDPs/IDRs), which are closely related to low complexity sequences and prion-like domains. The terms “intrinsically disordered protein,” “intrinsically disordered region,” and “intrinsically disordered protein region” are used interchangeably. The term “Intrinsically disordered region (IDR),” as used herein refers to a protein region that exhibit considerable conformational heterogeneity. The biased amino acid sequences of DDRs encode an intrinsic preference for conformational disorder and an inability to fold into singular well-defined 3D structures under physiological conditions. For example, RNA binding proteins often contain IDRs with the sequence composition biased toward amino acids including R, G, S, and Y, which comprise sequences that appear to be necessary and sufficient for driving condensation into liquid-like protein droplets. The role of IDR/LCSs has been implicated in both liquid-like physiological assemblies and pathological protein aggregates.

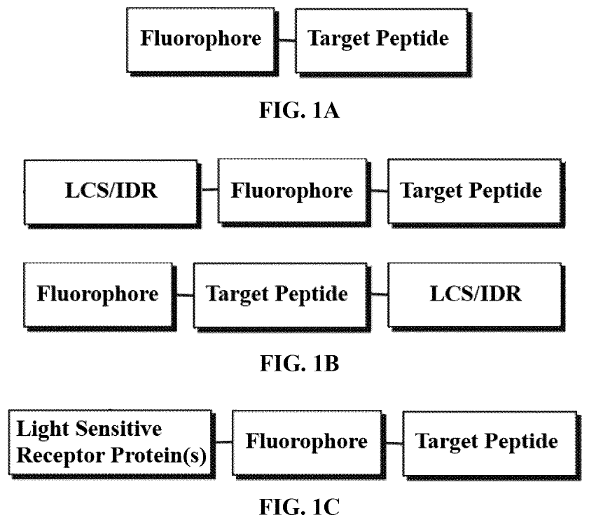

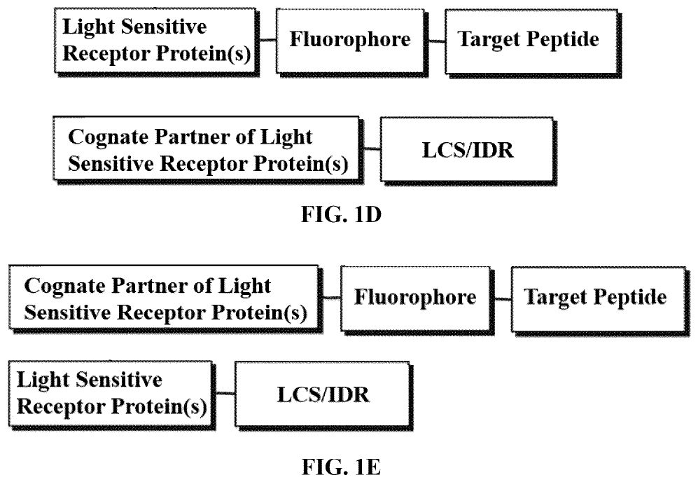

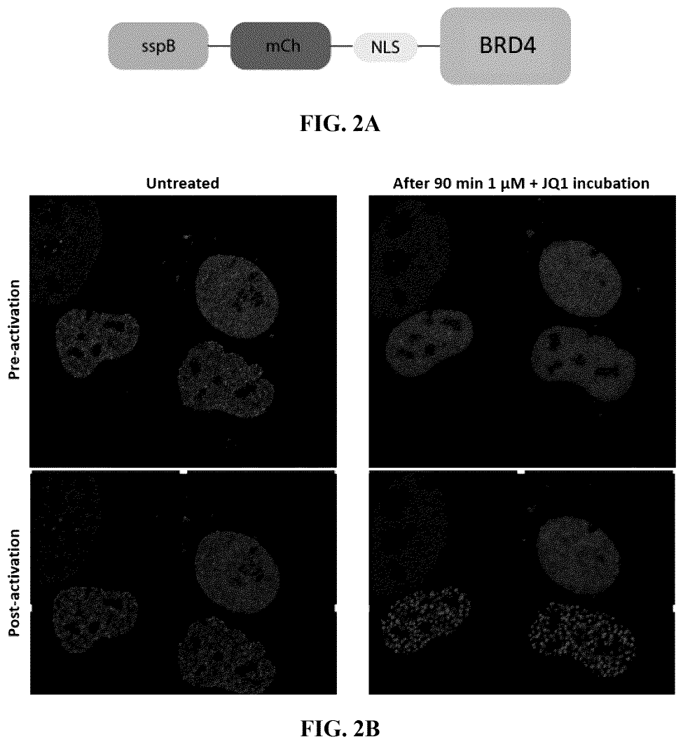

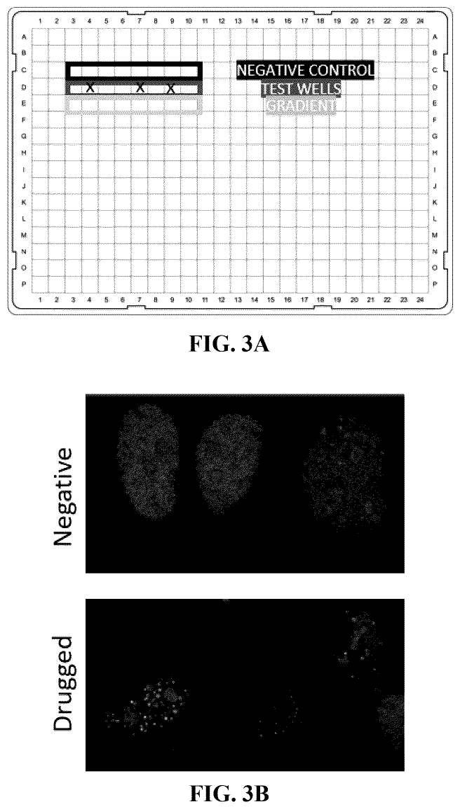

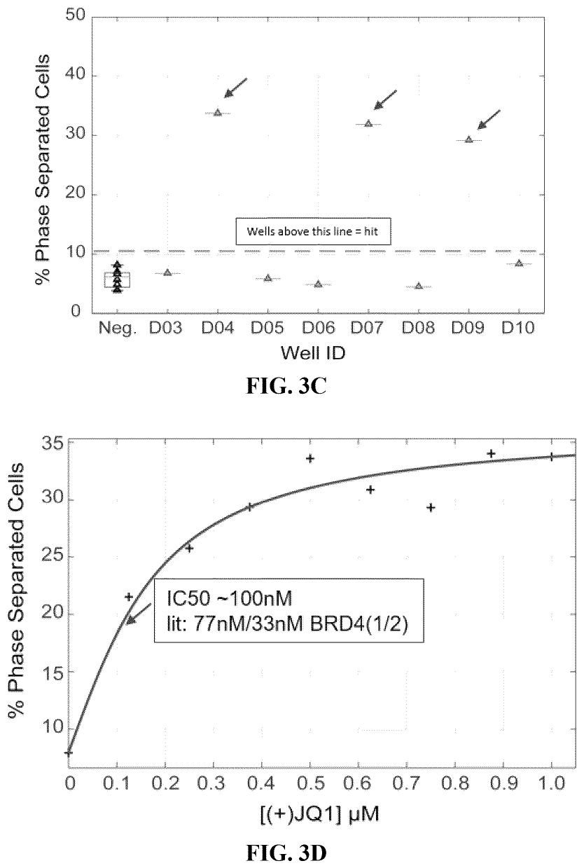

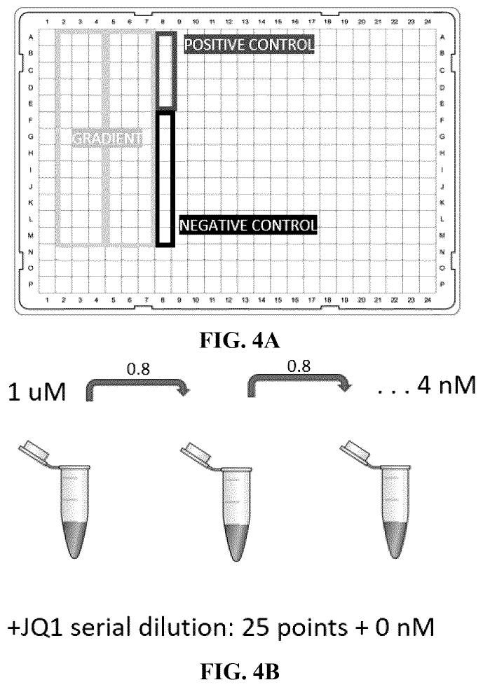

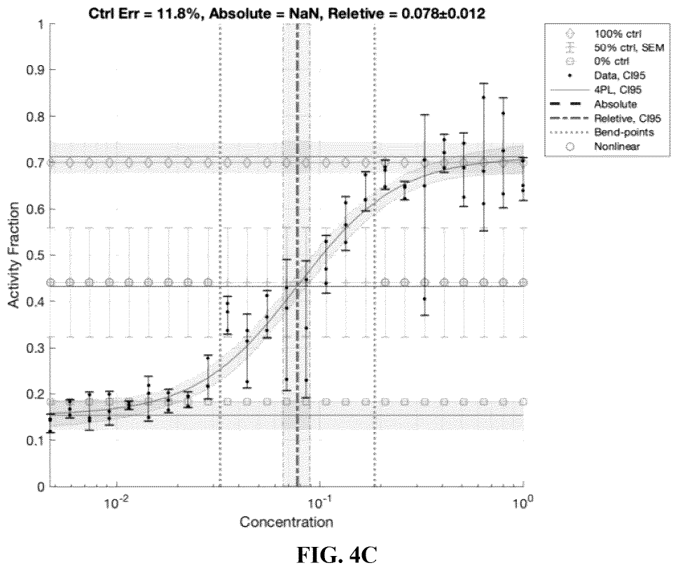

In some embodiments, the target peptide is an intrinsically disordered protein (IDP) or the target peptide comprises at least one intrinsically disordered region (IDR) (e.g., FIG. 1A and FIG. 1C). In some embodiments, the target molecule comprises additional IDR either fused to fluorophore or the target peptide to boost phase separation. In some embodiments, the additional IDRs are same as the IDRs of the target peptide. In some embodiments, the additional IDRs are different from the IDRs of the target peptide. In some embodiments, the target peptide is not an IDP nor the target peptide comprises an IDR.

In some embodiments, the functional region utilizes other proteins, such as synthetic or natural nucleic acid binding domains. Many RNA binding proteins contain self-associating IDRs or LCSs that can drive phase separation. However, in some embodiments, additional RNA binding domains can enhance phase separation via multivalent interactions with RNA. For example, FUS is an ALS-related RNA binding protein involved in diverse nucleic acid processing including DNA repair, transcription and pre-mRNA splicing. While the self-associating N-terminal IDR of FUS has been shown to be necessary and sufficient for liquid-liquid phase separation, C-terminal RNA binding domains appear to further promote phase separation. In some embodiments, the synthetic or natural nucleic acid binding domains utilize RNA recognition motifs (RRM), double-stranded RNA binding domains (dsRBD), S1, zinc finger binding domains, YT521-B homologies (YTH), DNA and RNA helicase domains, Pumilio, or S-adenosylmethionine (SAM) structures.

Exemplary IDPs include, but are not limited to full length or truncated forms of FUS (SEQ ID NO: 3), full length or truncated forms of DDX4 (SEQ ID NO: 4), full length or truncated forms of hnRNPA1 (SEQ ID NO: 2 or SEQ ID NO: 5) and other IDR-containing RNA binding domains. In some embodiments, the IDR comprises amino acid residues 1-214 of FUS, 1-236 of DDX4, or 186-320 of hnRNPA1. In some embodiments, the C-terminal IDR of the ALS-related RNA binding protein hnNRNPA1, or the N-terminal IDR of Ddx4 (optoDDX4) may be used to drive liquid-liquid phase separation. In some embodiments, the IDR of human FUS (residues 1-214), the IDR of human hnRNPA1 (residues 186-320), or the IDR of human DDX4 (residues 1-236) may be used to drive liquid-liquid phase separation. In some embodiments, a broad range of different IDR-containing proteins that display a remarkable diversity of their physical characteristics may be used. For example, in some embodiments, IDRs that range from relatively uncharged (e.g., TAF15 N) to highly basic (e.g. SRSF2 IDR) and mixed-charge (SART1); from relatively hydrophobic (e.g. HSF1) to highly hydrophilic (e.g. RNPS1) are used.

In some embodiments, exemplary LLPS-driven bodies or intracellular condensates include, but are not milited to nucleoli, Cajal bodies, stress granules, P-bodies and spliceosomes.

The term “nucleolus” or “nucleoli,” as used herein, refers to the most prominent nuclear body of eukaryotic cells. The nucleolus serves a fundamentally important biological role as a site of ribonucleoprotein particle assembly, primarily dedicated to ribosome biogenesis. The nucleolus is physically separated from the rest of the nuclear space, yet is accessible for dynamic exchange due to the absence of a delimiting membrane. It appears that the nucleolus is a multilayered biomolecular condensate, whose formation by LLPS facilitates the initial steps of ribosome biogenesis and other functions.

The term “Cajal bodies” or “coiled bodies,” as used herein, refers to nuclear condensates containing coilin and survival of motor neuron protein (SMN). Cajal bodies are membrane-less organelles and largely consist of proteins and RNA. Cajal bodies are enriched in U snRNAs and share some protein components with the nucleolus, such as fibrillarin. Cajal bodies have been implicated in RNA-related metabolic processes such as the biogenesis, maturation and recycling of snRNPs, histone mRNA processing and telomere maintenance. Cajal bodies assemble RNA, which is used by telomerase to add nucleotides to the ends of telomeres.

The term “stress granule,” as used herein, refers to cytoplasmic condensates that form in response to stress (for example, oxidative stress and heat stress). Stress granules are dense aggregations composed of proteins and RNAs. Stress granules are not surrounded by membrane, and associated with the endoplasmatic reticulum, that there are also nuclear stress granules.

The term “P body,” as used herein, refers to cytoplasmic condensates involved in mRNA degradation. P body are distinct foci formed by phase separation consisting of many enzymes involved in mRNA turnover. P-bodies appear to play fundamental roles in general mRNA decay, nonsense-mediated mRNA decay, adenylate-uridylate-rich element mediated mRNA decay, and miRNA induced mRNA silencing.

The term “spliceosome,” as used herein, refers to a large and complex molecular RNA identity found primarily within the nucleus of eukaryotic cells. Each spliceosome is composed of five small nuclear RNAs (snRNA) and a range of associated protein factors. When these small RNAs are combined with the protein factors, they make RNA-protein complexes called snRNPs (small nuclear ribonucleo proteins). The spliceosome removes introns from a transcribed pre-mRNA (splicing).

### Components for Screening Methods

### Cell

The methods of identifying an inhibitory activity of an exogeneous agent that disrupts binding of a target peptide to a biomolecule use cells comprising a target molecule, a target molecule and a second molecule, or a control molecule.

The term “cell,” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells.

In some embodiments, examples of suitable cells include, but are not limited to, a bacterial cell; an archaeal cell; a single-celled eukaryotic organism; a plant cell; an algal cell; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium falciparum; a helminth; etc.); a cell from a vertebrate animal (e.g., fish, amphibian, reptile, bird, mammal); a mammalian cell, e.g., a rodent cell, a human cell, a non-human primate cell, etc. Suitable host cells include naturally-occurring cells; genetically modified cells (e.g., cells genetically modified in a laboratory); and cells manipulated in vitro in any way. In some cases, a host cell is isolated or cultured.

In some embodiments, examples of cells include, but are not limited to, lung cells, heart cells, brain cells, microglia, blood cells, stomach cells, liver cells, intestinal cells, pancreatic cells, colon cells, kidney cells, ureter cells, bladder cells, lymphatic cells, leukocytes, muscle cells, neuronal cells, macrophages, astrocytes, stromal cells, spinal cord cells, ovarian cells, vagina cells, prostate cells, bone cells, cartilage cells, ligament cells, tendon cells, immune cells, hematopoietic cells, fibroblasts, macrophages, T cells, etc.

In some embodiments, the cell is a mammalian cell. In some embodiments, the cell is a cancer cell. In some embodiments, the cell is a cancer cell line. In some embodiments, the cancer cell is from a stomach cancer, esophageal cancer, head and neck squamous cell carcinoma (HNSCC), non-small cell lung cancer (NSCLC), and colorectal cancer (such as HNPCC). In some embodiments, the cancer cell include, but are not limited to, a breast cancer cell, a colon cancer cell, a kidney cancer cell, a leukemia cell, a lung cancer cell, a melanoma cell, an ovarian cancer cell, a prostate cancer cell, a pancreatic cancer cell, a brain cancer cell, a liver cancer cell, a gastric cancer cell or a sarcoma cell.

In some embodiments. The cells are established and/or immortalized cell lines. Non-limiting exemplary cell lines include 3T3 cells, A549 cells, HeLa cells, HEK 293 cells, Jurkat cells, OK cells, Ptk2 cells, Vero cells, U2OS cells, NIH3T3 cells, and HEK293T cells. In some embodiments, any type of cell may be of interest (e.g., a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells,” “primary cell lines,” and “primary cultures” are used. The terms “primary cells,” “primary cell lines,” and “primary cultures” interchangeably herein to refer to cells and cells cultures that have been derived from a subject and allowed to grow in vitro for a limited number of passages, i.e. splittings, of the culture. For example, primary cultures include cultures that may have been passaged 0 times, 1 time, 2 times, 4 times, 5 times, 10 times, or 15 times, but not enough times go through the crisis stage. Primary cell lines can be are maintained for fewer than 10 passages in vitro.

In some embodiments, cells in which some factors that improve the quality of the exogenous agents identified through screening a library of potential agents are expressed are suitable. In some embodiments, cells that are most physiologically relevant to the specific target or disease-state are suitable. In some embodiments, cells that have been validated to be models of a disease are suitable.

### Light-Sensitive Receptor

In some embodiments, a target molecule comprises a target peptide and at least one peptide selected from a light-sensitive receptor and a chemical-sensitive receptor. In some embodiments, a target molecule comprises a target peptide, at least one peptide selected from a light-sensitive receptor and a chemical-sensitive receptor, and a fluorophore fused between the target peptide and the at least one peptide.

In some embodiments, a target molecule comprises a target peptide, and a peptide selected from a group consisting of a light-sensitive receptor, a chemical-sensitive receptor, a light-sensitive oligomerization protein, and a non-light sensitive dimerization module, and a second molecule comprising a cognate partner of the peptide selected from a group consisting of a light-sensitive receptor, a chemical-sensitive receptor, a light-sensitive oligomerization protein, and a non-light sensitive dimerization module fused to a full length or truncated low complexity protein region or a full length or truncated intrinsically-disordered protein region (IDR). In some embodiments, a target molecule comprises a target peptide, a peptide selected from a group consisting of a light-sensitive receptor, a chemical-sensitive receptor, a light-sensitive oligomerization protein, and a non-light sensitive dimerization module, and a fluorophore fused between the target peptide and the peptide selected from a group consisting of a light-sensitive receptor, a chemical-sensitive receptor, a light-sensitive oligomerization protein, and a non-light sensitive dimerization module.

In some embodiments, a target molecule comprises a target peptide, and a cognate partner of a peptide selected from a group consisting of a light-sensitive receptor, a chemical-sensitive receptor, a light-sensitive oligomerization protein, and a non-light sensitive dimerization module, and a second molecule comprising the peptide selected from a group consisting of a light-sensitive receptor, a chemical-sensitive receptor, a light-sensitive oligomerization protein, and a non-light sensitive dimerization module fused to a full length or truncated low complexity protein region or a full length or truncated intrinsically-disordered protein region (IDR). In some embodiments, a target molecule comprises a target peptide, a cognate partner of a peptide selected from a group consisting of a light-sensitive receptor, a chemical-sensitive receptor, a light-sensitive oligomerization protein, and a non-light sensitive dimerization module, and a fluorophore fused between the target peptide and the cognate partner of the peptide selected from a group consisting of a light-sensitive receptor, a chemical-sensitive receptor, a light-sensitive oligomerization protein, and a non-light sensitive dimerization module.

In some embodiments, cells are exposed to at least one predetermined wavelength of light, a chemical to which the chemical-sensitive receptor is sensitive, or a combination thereof. In some embodiments, exposed to at least one predetermined wavelength of light, a chemical to which the chemical-sensitive sensitive receptor is sensitive, or a combination thereof, changes the conformation of the peptide selected from a group consisting of a light-sensitive receptor, a chemical-sensitive receptor, a light-sensitive oligomerization protein, and a non-light sensitive dimerization module, thereby, inducing conformational stress, which boosts phase separation of the target peptide.

In some embodiments, the light-sensitive receptor is cryptochrome 2 (Cry2), a photolyase homology region of Cry2, phytochrome B (PhyB), PIF, a light oxygen voltage sensing (LOV) domain, Dronpa, or an improved light-induced dimer (iLID). In some embodiments, the light-sensitive receptor is ssrA and the cognate partner is sspB. In some embodiments, the light-sensitive receptor is Cry2 or iLID, and the at least one predetermined wavelength of light is 450 nm wavelength of light or 440 nm wavelength of light in junction with 488 nm wavelength of light. In some embodiments, the light-sensitive receptor is PhyB or PIF, and the at least one predetermined wavelength of light is 650 nm wavelength of light in junction with 750 nm wavelength of light.

In some embodiments, the protein sensitive to at least one wavelength of light comprises a protein that is sensitive to visible light. In some embodiments, the protein sensitive to at least one wavelength of light is Cry2, Cry2olig, PhyB, PIF, light-oxygen-voltage sensing (LOV) domains, or Dronpa. Exposing the living cells comprising the protein sensitive to at least one wavelength of light to certain wavelengths of light induces the protein within the living cell to cluster.

In some embodiments, the methods as described herein utilize a photo-activatable or photo-deactivatable interaction between a light sensitive receptor protein on a target molecule and its cognate partner on a second molecule or vice versa in order to control the recruitment of intrinsically disordered proteins into membrane-less bodies by phase separation. In some embodiments, as each of the target peptide is fused to a light sensitive receptor protein, light is used to trigger the assembly or possibly disassembly of a structure comprising the light sensitive receptor protein on the target molecule with a cognate partner of the light sensitive receptor protein on a second molecule, where the cognate partner is fused to a full length or truncated low complexity or intrinsically-disordered protein. In some embodiments, photo-inducible reversible heterodimerization between the self-assemblying units (e.g., part of the target molecule) and IDR units (e.g., part of the second molecule) utilizes, e.g., the engineered blue light sensitive receptor protein iLID and its cognate partner, sspB. In some embodiments, one or both of the target molecule and the second molecule may be advantageously attached to a fluorescent protein marker (e.g., fluorophore). Exposing a living cell that express both molecules to certain wavelengths of light induces molecules within the living cell to cluster or nucleate liquid phases, gels, or aggregates including, for example, pathological protein aggregates such as amyloid fibers. In some embodiments, once formed, the phase separated clusters remain in the cells independent of the presence or absence of light.

In some embodiments, the at least one light sensitive receptor protein comprises one or more similar or different proteins responsive to at least one wavelength of light, for example, a wavelength of light in the near UV, visible or infra-red regions, which are from about 350 nm to about 800 nm. In some embodiments, a wavelength of light to which light sensitive receptor protein is responsive to is between 450 nm and 495 nm. In some embodiments, the at least one light sensitive receptor protein is sensitive to visible light. The wavelength of light is predetermined, based on the specific wavelengths to which the light sensitive protein utilized in the constructs is responsive.

In some embodiments, the light sensitive protein is the engineered protein iLID, which consist of a modified LOV2 domain fused at its C terminus to an ssrA peptide. In some embodiments, the self-assembling protein subunit is fused to two or more LOV2-ssrA proteins. In some embodiments, other light sensitive proteins, e.g., Cry2, PhyB or a LOV2 domain fused to a signaling peptide other than ssrA, are utilized. In some embodiments, the second molecule comprises at least one cognate partner of the light sensitive receptor protein fused to a full length or truncated low complexity sequence (LCS) or IDR. In some embodiments, a fluorescent tag (e.g., fluorophore) is included in the second molecule, for example, between the cognate partner and the LCS or IDP or any other locations if desired.

In some embodiments, the cognate partner of the light sensitive receptor protein is any appropriate cognate of the light sensitive receptor protein. Exemplary cognate partners include, but are not limited to ssrB, Zdk, CIB, and PIF as a cognate partner for LOV2-ssrA, LOV2, Cry2, and PhyB, respectively. In some embodiments, the second molecule comprises an IDP.

In some embodiments, the light sensitive receptor protein of the target molecule is a LOV2-ssrA domain, and the second molecule comprises the cognate partner of the LOV2-ssrA domain, sspB, fused to full length or truncated low complexity or intrinsically-disordered protein. In the active state (e.g., upon photoactivation), the light sensitive receptor protein binds to the cognate partner of the light sensitive receptor protein. In this example, the buried ssrA peptide becomes uncaged, and exposed ssrA rapidly binds its cognate sspB partner. Because the cognate partner is bound to an LCS/IDR, the clustering of LCS/IDR leads to the formation of membrane-less bodies.

### Fluorophore

In some embodiments, the target peptide is fused to a fluorophore. In some embodiments, the target molecule comprises a fluorophore fused between the target peptide and the at least one peptide selected from a light-sensitive receptor and a chemical-sensitive receptor. In some embodiments, the target molecule comprises a fluorophore fused between the target peptide and the peptide selected from a group consisting of a light-sensitive receptor, a chemical-sensitive receptor, a light-sensitive oligomerization protein, and a non-light sensitive dimerization module. In some embodiments, the target molecule comprises a fluorophore fused between the target peptide and the cognate partner of the peptide selected from a group consisting of a light-sensitive receptor, a chemical-sensitive receptor, a light-sensitive oligomerization protein, and a non-light sensitive dimerization module.

In some embodiments, the second molecule comprising a cognate partner of a peptide selected from a group consisting of a light-sensitive receptor, a chemical-sensitive receptor, a light-sensitive oligomerization protein, and a non-light sensitive dimerization module fused to a full length or truncated low complexity protein region or a full length or truncated intrinsically-disordered protein region (IDR) further comprises a fluorophore. In some embodiments, the fluorophore are fused between the cognate partner of a peptide selected from a group consisting of a light-sensitive receptor, a chemical-sensitive receptor, a light-sensitive oligomerization protein, and a non-light sensitive dimerization module, and a full length or truncated low complexity protein region or a full length or truncated intrinsically-disordered protein region (IDR). In some embodiments, the second molecule comprising a peptide selected from a group consisting of a light-sensitive receptor, a chemical-sensitive receptor, a light-sensitive oligomerization protein, and a non-light sensitive dimerization module fused to a full length or truncated low complexity protein region or a full length or truncated intrinsically-disordered protein region (IDR) further comprises a fluorophore. In some embodiments, the fluorophore are fused between the peptide selected from a group consisting of a light-sensitive receptor, a chemical-sensitive receptor, a light-sensitive oligomerization protein, and a non-light sensitive dimerization module, and a full length or truncated low complexity protein region or a full length or truncated intrinsically-disordered protein region (IDR). In some embodiments, the second molecule comprises a fluorophore that is different from the fluorophore that the target molecule comprises.

The term “fluorophore” or “fluorochrome,” as used herein, refers to a fluorescent chemical compound that can re-emit light upon light excitation. Fluorophores typically contain several combined aromatic groups, or planar or cyclic molecules with several π bonds. The exemplary fluorophores include, but are not limited to, the fluorophores listed in Table 1. In some embodiments, the target molecule as described herein comprise any one of the fluorophores listed in Table 1.

TABLE 1

| | | | |
|---|---|---|---|
| | Excitation | Emission | |
| | wave- | wave- | |
| | length | length | |
| Dye | (nm) | (nm) | Notes |

Reactive and conjugated dyes

| | | | |
|---|---|---|---|
| Hydroxycoumarin | 325 | 386 | Succinimidyl ester |
| Aminocoumarin | 350 | 445 | Succinimidyl ester |
| Methoxycoumarin | 360 | 410 | Succinimidyl ester |
| Cascade Blue | (375); 401 | 423 | Hydrazide |
| Pacific Blue | 403 | 455 | Maleimide |
| Pacific Orange | 403 | 551 | |
| Lucifer yellow | 425 | 528 | |
| NBD | 466 | 539 | NBD-X |
| R-Phycoerythrin (PE) | 480; 565 | 578 | |
| PE-Cy5 conjugates | 480; 565; 650 | 670 | Cychrome, R670, |
| | | | Tri-Color, |
| | | | Quantum Red |
| PE-Cy7 conjugates | 480; 565; 743 | 767 | |
| Red 613 | 480; 565 | 613 | PE-Texas Red |
| PerCP | 490 | 675 | Peridinin chlorophyll |
| | | | protein |
| TruRed | 490,675 | 695 | PerCP-Cy5.5 |
| | | | conjugate |
| FluorX | 494 | 520 | (GE Healthcare) |
| Fluorescein | 495 | 519 | FITC; pH sensitive |
| BODIPY-FL | 503 | 512 | |
| G-Dye100 | 498 | 524 | |
| G-Dye200 | 554 | 575 | |
| G-Dye300 | 648 | 663 | |
| G-Dye400 | 736 | 760 | |
| Cy2 | 489 | 506 | QY 0.12 |
| Cy3 | (512); 550 | 570; (615) | QY 0.15 |
| Cy3B | 558 | 572; (620) | QY 0.67 |
| Cy3.5 | 581 | 594; (640) | QY 0.15 |
| Cy5 | (625); 650 | 670 | QY 0.28 |
| Cy5.5 | 675 | 694 | QY 0.23 |
| Cy7 | 743 | 767 | QY 0.28 |
| TRITC | 547 | 572 | TRITC |
| X-Rhodamine | 570 | 576 | XRITC |
| Lissamine Rhodamine B | 570 | 590 | |
| Texas Red | 589 | 615 | Sulfonyl chloride |
| Allophycocyanin (APC) | 650 | 660 | |
| APC-Cy7 conjugates | 650; 755 | 767 | Far Red |

Nucleic acid dyes

| | | | |
|---|---|---|---|
| 7-AAD | 546 | 647 | 7-aminoactinomycin |
| | | | D, CG-selective |
| Acridine Orange | 503 | 530/640 | DNA/RNA |
| Chromomycin A3 | 445 | 575 | CG-selective |
| CyTRAK Orange | 520 | 615 | (Biostatus) (red |
| | | | excitation dark) |
| DAPI | 345 | 455 | AT-selective |
| DRAQ5 | 600/647 | 697 | (Biostatus) |
| | | | (usable excitation |
| | | | down to 488) |
| DRAQ7 | 599/644 | 694 | (Biostatus) |
| | | | (usable excitation |
| | | | down to 488) |
| Ethidium Bromide | 210; 285 | 605 | in aqueous solution |
| Hoechst 33258 | 345 | 478 | AT-selective |
| Hoechst 33342 | 343 | 483 | AT-selective |
| LDS 751 | 543; 590 | 712; 607 | DNA (543ex/ |
| | | | 712em), RNA |
| | | | (590ex/607em) |
| Mithramycin | 445 | 575 | |
| Propidium Iodide (PI) | 536 | 617 | |
| SYTOX Blue | 431 | 480 | DNA |
| SYTOX Green | 504 | 523 | DNA |
| SYTOX Orange | 547 | 570 | DNA |
| Thiazole Orange | 510 | 530 | |
| TO-PRO: Cyanine | | | |
| Monomer | | | |
| TOTO-1, TO-PRO-1 | 509 | 533 | Vital stain, TOTO: |
| | | | Cyanine Dimer |
| TOTO-3, TO-PRO-3 | 642 | 661 | |
| YOYO-1 | 491 | 509 | |

Cell function dyes

| | | | |
|---|---|---|---|
| DCFH | 505 | 535 | 2′7′Dichorodi- |
| | | | hydrofluorescein, |
| | | | oxidized form |
| DHR | 505 | 534 | Dihydrorhodamine |
| | | | 123, oxidized |
| | | | form, light |
| | | | catalyzes oxidation |
| Fluo-3 | 506 | 526 | AM ester. pH >6 |
| Fluo-4 | 491/494 | 516 | AM ester. pH 7.2 |
| Indo-1 | 361/330 | 490/405 | AM ester, low/high |
| | | | calcium (Ca²⁺) |
| SNARF | 548/579 | 587/635 | pH 6/9 |

Fluorescent proteins

| | | | |
|---|---|---|---|
| Allophycocyanin (APC) | 652 | 657.5 | heterodimer, |
| | | | crosslinked |
| AmCyan1 | 458 | 489 | tetramer |
| AsRed2 | 576 | 592 | tetramer |
| Azami Green | 492 | 505 | monomer |
| Azurite | 383 | 447 | monomer |
| B-phycoerythrin (BPE) | 545 | 572 | heterotrimer |
| Cerulean | 433 | 475 | weak dimer |
| CyPet | 435 | 477 | weak dimer |
| DsRed monomer | 556 | 586 | monomer |
| DsRed2 (″RFP″) | 563 | 582 | |
| EBFP | 380 | 440 | monomer |
| EBFP2 | 383 | 448 | monomer |
| ECFP | 434 | 477 | |
| EGFP | 488 | 507 | weak dimer |
| Emerald | 487 | 509 | weak dimer |
| EYFP | 514 | 527 | weak dimer |
| GFP (S65A mutation) | 471 | 504 | |
| GFP (S65C mutation) | 479 | 507 | |
| GFP (S65L mutation) | 484 | 510 | |
| GFP (S65T mutation) | 488 | 511 | |
| GFP (Y66F mutation) | 360 | 508 | |
| GFP (Y66H mutation) | 360 | 442 | |
| GFP (Y66W mutation) | 436 | 485 | |
| GFPuv | 385 | 508 | |
| HcRed1 | 588 | 618 | dimer |
| J-Red | 584 | 610 | dimer |
| Katusha | 588 | 635 | dimer |
| Kusabira Orange | 548 | 559 | monomer |
| mCFP | 433 | 475 | monomer |
| mCherry | 587 | 610 | monomer |
| mCitrine | 516 | 529 | monomer |
| Midoriishi Cyan | 472 | 495 | dimer |
| mKate (TagFP635) | 588 | 635 | monomer |
| mKeima-Red | 440 | 620 | monomer |
| mKO | 548 | 559 | monomer |
| mOrange | 548 | 562 | monomer |
| mPlum | 590 | 649 | |
| mRaspberry | 598 | 625 | monomer, faster |
| | | | photobleach |
| | | | than mPlum |
| mRFP1 | 584 | 607 | monomer |
| mStrawberry | 574 | 596 | monomer |
| mTFP1 | 462 | 492 | dimer |
| m Turquoise2 | 434 | 474 | monomer |
| P3 | 614 | 662 | phycobilisome |
| | | | complex |
| Peridinin Chlorophyll | 483 | 676 | trimer |
| (PerCP) | | | |
| R-phycoerythrin (RPE) | 565 > 498 | 573 | heterotrimer |
| T-Sapphire | 399 | 511 | weak dimer |
| TagCFP | 458 | 480 | dimer |
| TagGFP | 482 | 505 | monomer |
| TagRFP | 555 | 584 | monomer |
| Tag YFP | 508 | 524 | monomer |
| tdTomato | 554 | 581 | tandem dimer |
| Topaz | 514 | 527 | monomer |
| TurboFP602 | 574 | 602 | dimer |
| TurboFP635 | 588 | 635 | dimer |
| TurboGFP | 482 | 502 | dimer |
| TurboRFP | 553 | 574 | dimer |
| Turbo YFP | 525 | 538 | dimer |
| Venus | 515 | 528 | weak dimer |
| Wild Type GFP | 396, 475 | 508 | |
| YPet | 517 | 530 | weak dimer |
| ZsGreen 1 | 493 | 505 | tetramer |
| ZsYellow1 | 529 | 539 | tetramer |

### Pharmaceutical Compositions

Pharmaceutical compositions or formulations comprising the agents, e.g., the agents that disrupts binding of a target peptide to a biomolecule as screened by the methods as described herein, for use in the methods of treatment as described herein can be prepared according to conventional techniques well known in the pharmaceutical industry and described in the published literature. In embodiments, a pharmaceutical composition or formulation for treating a subject comprises an effective amount of the agents as described herein. The pharmaceutical formulation comprising the agents as described herein may further comprise a pharmaceutically acceptable excipient, diluent or carrier.

The pharmaceutical composition or formulation of the present invention may comprise one or more penetration enhancer, carrier, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present invention employs a penetration enhancer to effect the efficient delivery of the agent, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

The term “preparation,” is intended to include the formulation of the compositions with encapsulating material as a carrier providing a capsule in which the active compositions with or without other carriers, is surrounded by a carrier, which is thus in association with it.

The administration of compositions described herein can be carried out in any convenient manner, including by aerosol inhalation, injection, ingestion, transfusion, implantation or transplantation. Examples of routes of administration include parenteral, e.g., intravenous or intra-arterial, intradermal, subcutaneous, oral (e.g., inhalation), transdermal (topical), transmucosal, nasal, pulmonary, ocular, gastrointestinal, and rectal administration. Alternate routes of administration include intraperitoneal, intra-articular, intracardiac, intracisternal, intradermal, intralesional, intraocular, intrapleural, intrathecal, intrauterine, intraventricular, and the like.

For oral administration, the compositions can be formulated in liquid or solid dosage forms and as instant or controlled/sustained release formulations. Suitable dosage forms for oral ingestion by a subject include powders, tablets, pills, granules, dragees, hard and soft shell capsules, liquids, gels, syrups, slurries, suspensions, emulsions and the like. Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, granules, pills, capsules, troches and the like can contain any of the following ingredients, or compounds of a similar nature: a binder such as micro-crystalline cellulose, gum tragacanth or gelatin; an excipient such as starch or lactose; dissolution retardant; anti-adherants; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

In one embodiment, the compositions are prepared with carriers that will protect the components of the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral anti-gens) can also be used as pharmaceutically acceptable carriers. These can be prepared according to methods known to those skilled in the art, for example, as described in U.S. Pat. No. 4,522,811.

For administration by inhalation, the compositions are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The compositions may also be formulated in rectal compositions, such as suppositories (e.g., with conventional suppository bases such as cocoa butter and other glycerides) or retention enemas.

Pharmaceutical compositions suitable for injectable use include sterile aqueous solutions (where water soluble) or dispersions and sterile powders for the extemporaneous preparation of sterile injectable solutions or dispersion. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. For example, depending on the injection site, the vehicle may contain water, synthetic or vegetable oil, and/or organic co-solvents. In certain instances, such as with lyophilized product or a concentrate, the parenteral formulation would be reconstituted or diluted prior to administration. pH can be adjusted with acids or bases, such as hydrochloric acid or sodium hydroxide. Depot formulations, providing controlled or sustained release of a composition, may include injectable suspensions of nano/micro particles or nano/micro or non-micronized crystals.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, poly(ol) (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in an appropriate solvent with one or a combination of ingredients enumerated above, as required, followed by filtered sterilization. Prevention of the action of micro-organisms can be achieved by various antibacterial and antifungal agents, for example, parabens, chlorobutanol, phenol, ascorbic acid, thimerosal, and the like.

Generally, dispersions are prepared by incorporating the active composition into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin. The parenteral preparation can be enclosed in ampoules, disposable syringes or multiple dose vials made of glass or plastic

Examples of pharmaceutically or physiologically acceptable carriers, diluents or excipients include, but are not limited to, antifoaming agents, antioxidants, binders, carriers or carrier materials, dispersing agents, viscosity modulating agents, diluents, filling agents, lubricants, glidants, plasticizers, solubilizers, stabilizers, suspending agents, surfactants, viscosity enhancing agents, and wetting agents. Supplementary active agents can also be incorporated into the compositions. Carrier molecules may be genes, polypeptides, antibodies, liposomes or indeed any other agent provided that the carrier does not itself induce toxicity effects or cause the production of antibodies that are harmful to the individual receiving the pharmaceutical composition. Further examples of known carriers include polysaccharides, polylactic acids, polyglycolic acids and inactive virus particles.

Carriers may also include pharmaceutically acceptable salts such as mineral acid salts (for example, hydrochlorides, hydrobromides, phosphates, sulphates) or the salts of organic acids (for example, acetates, propionates, malonates, benzoates). Pharmaceutically acceptable carriers may additionally contain liquids such as water, saline, glycerol, ethanol or auxiliary substances such as wetting or emulsifying agents, pH buffering substances and the like. Carriers may enable the pharmaceutical or nutraceutical compositions to be formulated into tablets, pills, dragees, capsules, liquids, gels, syrups, slurries, suspensions to aid intake by the patient.

“Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. Various formulations and drug delivery systems are available in the art, and a thorough discussion of pharmaceutically acceptable carriers are available in the art. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Also, the separate components of the pharmaceutical compositions as described herein may be preblended or each component may be added separately to the same environment according to a predetermined dosage for the purpose of achieving the desired concentration level of the treatment components and so long as the components eventually come into intimate admixture with each other. Further, the pharmaceutical compositions as described herein may be administered or delivered on a continuous or intermittent basis.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In embodiments, the treatment is only on an as-needed basis, e.g., upon appearance of signs or symptoms of a disease or a disorder.

For any composition described herein, the therapeutically effective amount can be initially determined from cell culture assays. Target concentrations will be those concentrations of active component(s) of the composition that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Toxicity and therapeutic efficacy of the pharmaceutical compositions as described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

The amount of the compound in the composition should also be in therapeutically effective amounts. The phrase “therapeutically effective amounts” used herein refers to the amount of agent needed to treat, ameliorate, or prevent a targeted disease or condition. An effective initial method to determine a “therapeutically effective amount” may be by carrying out cell culture assays (for example, using neuronal cells) or using animal models (for example, mice, rats, rabbits, dogs or pigs). A dose may be formulated in animal models to achieve a concentration range that includes the IC50 (i.e., the concentration of the composition which achieves a half-maximal inhibition of symptoms) as determined in cell culture. Such information can be used to more accurately determine useful doses in humans. In addition to determining the appropriate concentration range for a composition to be therapeutically effective, animal models may also yield other relevant information such as preferable routes of administration that will give maximum effectiveness. Such information may be useful as a basis for patient administration. A “patient” as used in herein refers to the subject who is receiving treatment by administration of the compound of interest.

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the pharmaceutical composition as described herein used for treatment may increase or decrease over the course of a particular treatment. Changes in dosage may result and become apparent from the results of diagnostic assays as described herein. The therapeutically-effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

The methods and the pharmaceutical compositions as described herein including embodiments thereof can be administered with one or more additional therapeutic regimens or agents or treatments, which can be co-administered to the mammal. By “co-administering” is meant administering one or more additional therapeutic regimens or agents or treatments and the pharmaceutical compositions as described herein sufficiently close in time to enhance the effect of one or more additional therapeutic agents, or vice versa. In this regard, the pharmaceutical compositions as described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the pharmaceutical compositions as described herein.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the pharmaceutical compositions as described herein are dictated by and directly dependent on the unique characteristics of the compositions and the particular therapeutic effect to be achieved, and the limitations inherent in the art of compounding such an agent for the treatment of subjects.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

The terms “prevent,” “preventing,” and “prevention,” as used herein, refer to a decrease in the occurrence of pathology of a disease or a disorder as described herein in a subject. The prevention may be complete, e.g., the total absence of pathology of a disease or a disorder in a subject. The prevention may also be partial, such that the occurrence of pathology a disease or a disorder in a subject is less than that which would have occurred without the methods of treatment as described herein.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

In some embodiments, exemplary diseases or disorders in a subject to be treated by the agents that disrupts binding of a target peptide to a biomolecule of which inhibitory activity is identified as described herein or the pharmaceutical composition comprising the same as described herein include, but are not limited to, cancer, an inflammatory disease, or a neurological disease.

For instance, examples of cancer, includes, but are not limited to, a malignant, pre-malignant or benign cancer. Cancers to be treated include, for example, a solid tumor, a lymphoma or a leukemia. In one embodiment, a cancer can be, for example, a brain tumor (e.g., a malignant, pre-malignant or benign brain tumor such as, for example, a glioblastoma, an astrocytoma, a meningioma, a medulloblastoma or a peripheral neuroectodermal tumor), a carcinoma (e.g., gall bladder carcinoma, bronchial carcinoma, basal cell carcinoma, adenocarcinoma, squamous cell carcinoma, small cell carcinoma, large cell undifferentiated carcinoma, adenomas, cystadenoma, etc.), a basalioma, a teratoma, a retinoblastoma, a choroidea melanoma, a seminoma, a sarcoma (e.g., Ewing sarcoma, rhabdomyosarcoma, craniopharyngeoma, osteosarcoma, chondrosarcoma, myosarcoma, liposarcoma, fibrosarcoma, leimyosarcoma, Askin's tumor, lymphosarcoma, neurosarcoma, Kaposi's sarcoma, dermatofibrosarcoma, angiosarcoma, etc.), a plasmocytoma, a head and neck tumor (e.g., oral, laryngeal, nasopharyngeal, esophageal, etc.), a liver tumor, a kidney tumor, a renal cell tumor, a squamous cell carcinoma, a uterine tumor, a bone tumor, a prostate tumor, a breast tumor including, but not limited to, a breast tumor that is Her2- and/or ER- and/or PR-, a bladder tumor, a pancreatic tumor, an endometrium tumor, a squamous cell carcinoma, a stomach tumor, gliomas, a colorectal tumor, a testicular tumor, a colon tumor, a rectal tumor, an ovarian tumor, a cervical tumor, an eye tumor, a central nervous system tumor (e.g., primary CNS lymphomas, spinal axis tumors, brain stem gliomas, pituitary adenomas, etc.), a thyroid tumor, a lung tumor (e.g., non-small cell lung cancer (NSCLC) or small cell lung cancer), a leukemia or a lymphoma (e.g., cutaneous T-cell lymphomas (CTCL), non-cutaneous peripheral T-cell lymphomas, lymphomas associated with human T-cell lymphotrophic virus (HTLV) such as adult T-cell leukemia/lymphoma (ATLL), B-cell lymphoma, acute non-lymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma, etc.), a multiple myeloma, a skin tumor (e.g., basal cell carcinomas, squamous cell carcinomas, melanomas such as malignant melanomas, cutaneous melanomas or intraocular melanomas, Dermatofibrosarcoma protuberans, Merkel cell carcinoma or Kaposi's sarcoma), a gynecologic tumor (e.g., uterine sarcomas, carcinoma of the fallopian tubes, carcinoma of the endometrium, carcinoma of the cervix, carcinoma of the vagina, carcinoma of the vulva, etc.), Hodgkin's disease, a cancer of the small intestine, a cancer of the endocrine system (e.g., a cancer of the thyroid, parathyroid or adrenal glands, etc.), a mesothelioma, a cancer of the urethra, a cancer of the penis, tumors related to Gorlin's syndrome (e.g., medulloblastomas, meningioma, etc.), a tumor of unknown origin; or metastases of any thereto. In some embodiments, the cancer is a lung tumor, a breast tumor, a colon tumor, a colorectal tumor, a head and neck tumor, a liver tumor, a prostate tumor, a glioma, glioblastoma multiforme, a ovarian tumor or a thyroid tumor; or metastases of any thereto. In some other embodiments, the cancer is an endometrial tumor, bladder tumor, multiple myeloma, melanoma, renal tumor, sarcoma, cervical tumor, leukemia, and neuroblastoma.

In some embodiments, the inflammatory disorder partially or fully results from obesity, metabolic syndrome, an immune disorder, an neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a protozoal infection, a viral infection, a fungal infection, or combinations thereof. In some embodiments, the inflammatory disorder is Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenia purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjëgren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA); or combinations thereof.

In some embodiments, examples of the neurological disease include, but are not limited to, Aarskog syndrome, Alzheimer's disease, amyotrophic lateral sclerosis (Lou Gehrig's disease), aphasia, Bell's Palsy, Creutzfeldt-Jakob disease, cerebrovascular disease, Cornelia de Lange syndrome, epilepsy and other severe seizure disorders, dentatorubral-pallidoluysian atrophy, fragile X syndrome, hypomelanosis of Ito, Joubert syndrome, Kennedy's disease, Machado-Joseph's diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere's disease, Parkinson's disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William's syndrome.

In some embodiments, a method of screening an agent that reduces or inhibits formation of membrane-less bodies driven by phase separation at a sub-region of a cell is contemplated. In some embodiments, a method of screening an agent that reduces or inhibits the number of membrane-less bodies driven by phase separation at a sub-region of a cell is also contemplated. In some embodiments, the agent is an exogeneous agent. In some embodiments, the membrane-less bodies are nucleoli, Cajal bodies, Stress granule, P-bodies, spliceosomes, or a combination thereof. In some embodiments, the agent is a small molecule compound. In some embodiments, the small molecule compound is an organic compound or an inorganic compound. In some embodiments, the agent is selected from the group consisting of a nucleic acid, a polynucleotide, a polypeptide, or a combination thereof. In some embodiments, the polypeptide is an antibody or a functional fragment thereof. In some embodiments, the polynucleotide is an aptamer, a RNA-based compound, an antisense nucleic acid, a PNA, or an combination thereof. In some embodiments, the RNA-based compound is a small interfering RNA, a microRNA, a small hairpin RNA, or a combination thereof. In some embodiments, the agent is a therapeutic agent. In some embodiments, a pharmaceutical composition comprising the agent, and a pharmaceutically acceptable excipient, diluent, or carrier is also contemplated. In some embodiments, a method of preparing the pharmaceutical composition is also contemplated. In some embodiments, described herein is a method of treating a disease or condition in a subject in need thereof, comprising administering the pharmaceutical composition, wherein the administering reduces a sign or a symptom associated with the disease or condition, thereby treating the disease or condition in the subject. In some embodiments, disease or condition is cancer, a neurodegenerative disorder, or an inflammatory disorder. In some embodiments, the subject is human, using LLPS to discover small molecules that can modulate protein interactions found in disease states.

### Modulating Cellular Interactions

Described herein, in some embodiments, is a method of modulating interaction between a target peptide and a biomolecule in a cell of a subject, the cell comprising a target molecule. In some embodiments, the target molecule comprises a target peptide. In some embodiments, the method comprises contacting a cell with an agent that disrupts binding of the target peptide to the biomolecule so as to release the target peptide from the biomolecule. In some embodiments, the method comprises incorporating the released target molecule into membrane-less bodies in the cell. In some embodiments, the released target molecule undergoes phase separation at a sub-region of the cell. In some embodiments, the sub-region of the cell is separate from a sub-region of the cell where the target peptide binds to the biomolecule. In some embodiments, the interaction between the target peptide and the biomolecule is modulated by detecting the membrane-less bodies comprising the target molecule. In some embodiments, the membrane-less bodies comprise the target molecule.

Described herein, in some embodiments, is a method of modulating subcellular localization of a target molecule in a cell of a subject. In some embodiments, the target molecule comprises a target peptide. In some embodiments, the method comprises contacting a cell with an agent that disrupts binding of the target peptide to the biomolecule so as to release the target peptide from the biomolecule. In some embodiments, the method comprises incorporating the released target molecule into membrane-less bodies. In some embodiments, the released target molecule undergoes phase separation at a sub-region of the cell. In some embodiments, the sub-region of the cell is separate and distinct from a sub-region where the target peptide binds to the biomolecule. In some embodiments, the interaction between the target peptide and the biomolecule is modulated by detecting the membrane-less bodies comprising the target molecule. In some embodiments, the membrane-less bodies comprise the target molecule.

Described herein, in some embodiments, is a method of determining effectiveness of a treatment regimen in a subject. In some embodiments, the method comprises treating the subject with an agent that disrupts binding of a target peptide to a biomolecule. In some embodiments, the method comprises forming membrane-less bodies in a cell from the subject. In some embodiments, the cell comprises the target molecule. In some embodiments, the target molecule comprises the target peptide. In some embodiments, the target molecule is incorporated into the membrane-less bodies when the target peptide is released from the biomolecule and undergoes phase separation at a sub-region of the cell that is separate from a sub-region where the target peptide binds to the biomolecule. In some embodiments, the method comprises determining effectiveness of the treatment regimen by detecting a change in an intensity or a size of the membrane-less bodies as compared to an intensity or size of membrane-less bodies in the absence of the agent.

In some embodiments, the target peptide comprises a fluorophore and detecting the membrane-less bodies is measured using fluorescence. In some embodiments, the target peptide is fused to a fluorophore. In some embodiments, a fluorophore is mCherry. In some embodiments, a fluorophore is green fluorescent protein (GFP). In some embodiments, measuring fluorescence comprises detecting an intensity of fluorescence, quantifying the number of the membrane-less bodies, or a combination thereof. In some embodiments, the intensity of fluorescence of the membrane-less bodies in the presence of an agent is higher by at least 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 10%, 20%, 30%, 40%, 50%, 60%, 70%, 80%, 90%, 100%, 200%, 300%, 400%, 500%, 600%, 700%, 800%, 900%, 1000%, 2000%, 3000%, 4000%, 5000%, 6000%, 7000%, 8000%, 9000%, 10000%, 20000%, 30000%, 40000%, 50000%, 60000%, 70000%, 80000%, 90000%, or 100000% as compared to an intensity of fluorescence of the membrane-less bodies in the absence of the agent.

In some embodiments, the target peptide is an intrinsically disordered protein (IDP) or the target peptide comprises at least one intrinsically disordered region (IDR) (e.g., FIG. 1A and FIG. 1C). In some embodiments, the target molecule comprises additional IDR either fused to fluorophore or the target peptide to boost phase separation. In some embodiments, the additional IDRs are same as the IDRs of the target peptide. In some embodiments, the additional IDRs are different from the IDRs of the target peptide. In some embodiments, the target peptide is not an IDP nor the target peptide comprises an IDR.

In some embodiments, a membrane-less body may be a nucleoli, Cajal body, stress granule, P-body, spliceosome, or a combination thereof.

In some embodiments, a biomolecule is a protein, a protein domain, DNA, RNA, a complex comprising a protein, or a combination thereof. In some embodiments, an agent is a small molecule. In some embodiments, a small molecule may be an organic molecule. In some embodiments, a small molecule may be an inorganic molecule. In some embodiments, an agent may be a nucleic acid, a polynucleotide, a polypeptide, or any combination thereof. In some embodiments, a polypeptide may be an antibody or a functional fragment thereof. In some embodiments, a polynucleotide may be an aptamer, an RNA-based compound, an antisense nucleic acid, an PNA, or a combination thereof. In some embodiments, the RNA-based compound is a small interfering RNA, a micro-RNA, a small hairpin RNA, or a combination thereof. In some embodiments, the polynucleotide is a CRISPR guide RNA. In some embodiments, a CRISPR guide RNA is encoded by a viral vector. In some embodiments, a viral vector may be a lentiviral vector.

In some embodiments, a CRISPR guide RNA may be introduced in combination with Cas9 or a vector comprising a nucleotide sequence encoding Cas9. In some embodiments, Cas9 is enzymatically dead Cas9 (e.g, dCas9).

In some embodiments, a target molecule may further comprise a full length complexity region fused to target peptide. In some embodiments, a target molecule may further comprise a truncated low complexity region fused to the target peptide. In some embodiments, a target molecule may further comprise an additional full length or truncated IDR fused to the target peptide. In some embodiments, the additional full length or truncated IDR is different from the target protein. In some embodiments, the additional full length or truncated IDR is the same as the target protein. In some embodiments, the IDR is at least a portion of FUS, a portion of Ddx4, a portion of hnRNPA1, a portion of BRD4, a portion of TAF15, a portion of SRSF2 IDR, a portion of SART1, a portion of HSF1, a portion of RNPS1, or a combination thereof. In some embodiments, the portion of FUS is an N-terminal IDR of FUS (e.g, FUSn).

In some embodiments, the target molecule may undergo phase separation as described elsewhere in the methods herein.

In some embodiments, the sub-region or subcellular region may be an organelle in a cell, such as nucleus, mitochondria, Golgi apparatus, ribosomes, lysosomes, endoplasmic reticulum, microtubules, or a combination thereof. In some embodiments, the sub-region or subcellular region may be a portion of the cell membrane, a portion of the nucleus, a portion of mitochondria, a portion of Golgi apparatus, a portion of ribosomes, a portion of lysosomes, a portion of endoplasmic reticulum, a portion of microtubules, or a combination thereof.

In some embodiments, a subject may be a mammal, for example a human or animal such as a non-human primate, a rodent, a rabbit, a rat, a mouse, a horse, a donkey, a goat, a cat, a dog, a cow, a pig, or a sheep. In some embodiments, the subject is a human. In other embodiments, the subject may be another eukaryotic organism, such as a plant.

The term “release,” or “released” as used herein, refers to breaking of the binding or interaction between a biomolecule and a target peptide. In some embodiments, a biomolecule binds to or interacts with a target peptide via dispersed variations of electromagnetic interactions between molecules or within a molecule, which include, but are not limited to, electrostatic interactions such as ionic interactions, a hydrogen bonding and a halogen bonding, van der Waals forces such as dipole-dipole interactions, dipole-induced dipole interactions and London dispersion forces, π-effects such as π-π interactions, cation-π and anion-π interactions and polar-π interactions, and hydrophobic effects. The term “release,” or “released” refer to weakening or breaking any one or more of the aforementioned interactions between the biomolecule and the target peptide.

### Kits

One aspect of the disclosure relates to kits to be used for the method of identifying an inhibitory activity of an exogeneous agent that disrupts binding of a target peptide to a biomolecule as described herein. The kits can further include one or more additional agents to be used for the method of identifying an inhibitory activity of an exogeneous agent that disrupts binding of a target peptide to a biomolecule as described herein.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific application. The label also indicates directions for use of the contents, such as in the methods described herein.

The present invention will be more specifically illustrated by the following Examples. However, it should be understood that the present invention is not limited by these examples in any manner.

### EXAMPLES

### Example 1

Presently, assays for the identification of inhibitors for disease-associated biomolecular interactions are most often performed in vitro and rely on extraction and/or purification of components combined with lengthy assay development and validation. In order to perform in cellulo screening, which is desirable for targeting interactions in their native environments and in the presence of their complete biochemical pathways, assays typically require years of development and validation. An exemplary embodiment of a technique that overcomes the requirement of years of development and validation is described below.

Using phase separation as a positive read-out for the change in binding status of a target protein offers an instantaneous signal that can be visually confirmed and further quantified by microscopy. This technique facilitates the in cellulo determination of the presence of inhibitors of disease target biomolecular interactions, where target interactions may include protein domains bound to modified histones, directly to DNA, or to other biomolecules/complexes. The presence of a successful inhibitor of the desired target interaction is indicated by a visible readout that can be measured on a confocal microscope. The readout is amenable to robust high-throughput screening with high z-score (e.g., >0.5) to identify hits from small molecule libraries, or possibly using genetic (CRISPR) screens or screens involving biologics.

The disclosed assay can be applied either to protein targets that naturally phase separate in the cell, or by adding a phase separating “tag” to the target. The phase separating “tag” can be any sequence that would be expected to cause the system to phase separate (such as an intrinsically disordered region (IDR)). Proof of concept requires a single experiment for each potential target, and once proof is established, because of the nature of the phase separation signal, the assay requires little optimization to transition to a robust screening platform with a high z-score. This assay is sensitive to the extent of inhibition as a function of extent of increase in phase separation.

Embodiments of the disclosed technique are based on characteristics of the “target” molecule that exhibits a binding site to be inhibited. The target protein is preferably expressed in tissue culture by viral incorporation into a cell genome or by CRISPR cell lines in the following scenarios:

If the target protein tends to phase separate “naturally,” it can be expressed in the cells as a fusion protein with a fluorophore such as mCh at an appropriate level. The positive read-out of phase separation can be observed by microscopy in the presence of potential inhibitor compounds. The extent of phase separation can be quantified by analyzing a population of cells expressing various concentrations of protein and determining the ratio of phase separating cells to the total population.

Phase separation in the above scenario can be augmented by optoDroplet, Corelet, or CasDrop systems (see, e.g., U.S. Ser. No. 15/618,345; U.S. Ser. No. 15/618,361; and WO 2019/147611 A2, all of which are incorporated herein by reference in their entireties). For example, the optoDroplet system was developed for controlling intracellular phase separation, by fusing the blue-light-dependent higher-order oligomerizing protein Cry2 to the IDR of DDX4. In some embodiments, using the Optodroplet system, the target protein is fused to a fluorophore, a light sensitive protein (such as Cry2), and a low complexity sequence (LCS) (see, e.g., FIG. 1C). For another example, the biomimetic Corelet system consists of a 24-mer ferritin core, where each ferritin subunit is fused to an optogenetic heterodimerization domain, which can be used to tune the effective valency of the particle with light.

This will allow for a highly sensitive and amplified signal to contrast between bound target protein and those liberated to participate in phase separation. Oligomerization would be initiated by a light at a wavelength appropriate for the light sensitive protein, the light being turned on for a period of time (e.g., 5 min) and phase separation will be enhanced in the presence of lead inhibitor compounds. As above, cells positive for phase separation will be identified by microscopy.

For target proteins that do not have the ability to “naturally” phase separate, protein fusions could be expressed by cloning the protein target with, e.g., an IDR tag, and then and optionally augmenting them with the optoDroplet, Corelet, or CasDrop systems as above.

This technique can be used to identify drugs that disrupt interactions between protein domains and chromatin (e.g. protein domains that bind to modified histones, for example as in the BRD4 bromodomains binding to acetylated lysines of histones), or protein domains that bind directly to DNA, for example basic helix loop helix domains in genetic repressors, etc., and/or interactions between protein domains and other biomolecules/complexes, for example protein domains that interact with cell surface receptors proteins, or other proteins or RNA, on or in the endoplasmic reticulum, Golgi apparatus, etc.

In the presence of a drug (e.g., from a library of small molecules) that disrupts those particular interactions, the protein would be liberated from sequestration at the location of its interaction partner. Once liberated, if the protein has a tendency to phase separate, which could be augmented by using the optoDroplet, Corelet, or CasDrop systems, this phase separation would be a powerful readout of the efficacy of a drug for disrupting the interaction.

### Example 2

An example of this technique is described as follows. The target protein was identified as BRD4, which binds to acetylated histone marks via two bromodomains. The compound+JQ1 is known to inhibit this interaction by binding to the two bromodomains with an IC50 of 77/33 nM BRD4(1/2).

While BRD4 falls into category of proteins that tend to separate “naturally”, an approach augmented with optogenetic components was utilized in this example. U2OS (and HEK293) cells (transiently) expressing the assay components: sspB-mCh-NLS-BRD4 and iLID-GFP-sspB (corelet system) were prepared. The polyclonal cell populations were transferred to wells of a 96-well plate. To prepare drugged cell populations, +JQ1 was added at 1 μM concentration and incubated for 90 min at 37° C. The BRD4 signal was measured by confocal microscopy by exciting the mCh fluorophore at 561 nm. The drugged and undrugged cell populations were compared by activating the corelet system with continuous laser light at 488 nm on a Nikon A1 confocal microscope. Upon activation, the drugged populations exhibited macroscopic phase separation indicating the liberation of BRD4 from sequestration at the histone marks. The undrugged populations remained sequestered. Using the extent of phase separation as a positive readout for the extent of BRD4 inhibition with +JQ1, an IC50 curve was constructed, and an IC50 of 78 nM was measured, in close agreement to the literature value.

“BRD4,” also known as Bromodomain-containing protein 4, CAP, HUNK1, HUNKI, MCAP, and bromodomain containing 4, as referred to herein, includes any of the recombinant or naturally-occurring forms of BRD4 protein or variants or homologs thereof that maintain BRD4 (e.g., within at least 40%50%, 60%, 70%, 80%, 90%, 95%, 96%, 97%, 98%, 99% or 100% activity compared to BRD4). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BRD4 protein. In some embodiments, the BRD4 protein is substantially identical to the protein identified by the UniProt reference number 060885 or a variant or homolog having substantial identity thereto. In some embodiments, BRD4 is a human BRD4 protein.

### Example 3

### Production of Cells Comprising a Target Molecule, or a Target Molecule and a Second Molecule

DNA constructs: DNA fragments encoding the target molecule and/or the second molecule are inserted into a lentiviral backbone. The resulting constructs are confirmed by, e.g., sequencing. These constructs are introduced into living cells.

Cell culture: several cells, for example, NIH3T3, HEK293, HEK293T, and U2OS cells, are cultured in growth medium consisting of Dulbecco's modified Eagle's medium, 10% fetal bovine serum, and 10 U/mL Penicillin-Streptomycin, and incubated at 37° C. and 5% CO₂ in a humidified incubator.

Lentiviral transduction: to produce stable cells expressing a target molecule, or a target molecule and a second molecule, lentivirus is produced by cotransfecting the transfer plasmids, pCMV-dR8.91, and pMD2.G (9:8:1, mass ratio) into HEK293T cells grown to approximately 70% confluency in multi-well, e.g., 6-well, plates using, e.g., FuGENE HD Transfection Reagent (Promega) per manufacturer's protocol. A total of, e.g., 3 ng plasmid and, e.g., 9 μL of transfection reagent are delivered into each well. Alternatively, lentiviral constructs are transfected into 293T cells that have been plated in multi-well, e.g., 6-well, dishes 1 day prior to the transfection using, e.g., FuGENE (Promega), following the manufacturer's recommended protocol. After 2 days, the supernatant containing viral particles is harvested and filtered with 0.45-μm filter to remove cell debris. The harvested supernatant is immediately used for transduction, concentrated 10-fold using Lenti-X Concentrator (Takara), or stored at −80° C. in aliquots. For lentiviral NIH3T3 or HEK293T cells are grown to 10-20% confluency in multi-well, e.g., 12-well, plates and 100-1000 μL of the filtered viral supernatant is added to the cells. Media containing virus is replaced with fresh growth medium 24 hours post-infection. Alternatively, NIH 3T3 cells plated at approximately 70% confluency in multi-well, e.g., 6-well, dishes are infected by directly adding 0.4-1 ml of filtered viral supernatant to the cell medium. Viral medium is replaced with normal growth medium 24 h after infection. Cells infected are typically imaged no earlier than 72 hours after infection.

Cell line generation: to establish cell lines expressing a target molecule, or a target molecule and a second molecule, sequential lentiviral transduction as performed, together with fluorescence activated cell sorting (FACS) when needed. Wild-type NIH3T3 or HEK293T cells are transduced with a lentivirus containing a sequence encoding a target molecule operatively linked to a promoter, a lentivirus containing a sequence encoding a target molecule and a sequence encoding a second molecule operatively linked to promoters, respectively, or a lentivirus containing a sequence encoding a target molecule operatively linked to a promoter together with a second lentivirus containing a sequence encoding a second molecule operatively linked to a promoter. This transduced NIH3T3 or HEK293T cell line is then used to generate other cell lines by lentiviral transduction to express other molecules. To enrich the cell population expressing a target molecule, or a target molecule and a second molecule in the transduced HEK293T cell line, cells are sorted on a FACSAria Fusion flow cytometer with gating for cells expressing a range of fluorescence. The polyclonal cell pool recovered in growth medium and plated.

Transient transfection: Cells, for example, HEK293, HEK293T, and U2OS cells, are grown to approximately 70% confluency in multi-well, e.g., 12-well, plates before being transfected with plasmid DNA using, e.g., Lipofectamine 3000 following manufacturer's protocol. Transfection reagents and DNA plasmids are diluted with OPTI-MEM (Gibco). Each well receives 100 μL of transfection mixture containing a total of 1 ng DNA. The transfection mixture is removed 6-24 hours post transfection. Cells transiently transfected are typically imaged between 24-48 hours post transfection.

Screening of Agents that Disrupts Binding of a Target Peptide to a Biomolecule

The cells expressing the assay components, i.e., a target molecule or a target molecule and a second molecule, e.g., U2OS cells or HEK293 cells are prepared. The control cells that express a control molecule (non-target molecule) are also prepared. The cells are transferred to wells of a multi-well, e.g., 96-well, plate. The library of agents to be screened for the inhibitory activity to disrupt binding of a target peptide to a biomolecule is added to each well of the multi-well plate containing the cells. The cells are incubated for a certain duration of time, e.g., 90 min, at 37° C. For the cells expressing a target molecule and a second molecule, the cells are exposed to a predetermined wavelength of light, e.g., blue light, to drive phase separation. The fluorescence is measured by confocal microscopy by exciting the fluorophore using a corresponding wavelength of light. The cells expressing a target molecule or a target molecule and a second molecule and treated with the agents, the cells expressing a target molecule or a target molecule and a second molecule and untreated, the cells expressing a control molecule and treated with the agents, the cells expressing a control molecule and untreated are compared by activating the fluorophore with continuous laser light at a corresponding wavelength on a Nikon A1 confocal microscope. Upon activation, the cells expressing a target molecule or a target molecule and a second molecule and treated with an agent having the inhibitory activity to disrupt binding of a target peptide to a biomolecule are expected to exhibit macroscopic phase separation, i.e., cluster assembly of the target molecule. By comparison, the cells expressing a target molecule or a target molecule and a second molecule and untreated, the cells expressing a control molecule and treated with the agents, the cells expressing a control molecule and untreated are expected not to exhibit macroscopic phase separation upon activation. Using the extent of phase separation as a positive readout for the inhibitory activity of the selected agent to disrupt binding of a target peptide to a biomolecule, an IC50 an IC50 is measured.

Immunocytochemistry: HEK293 cells expressing a target molecule, or a target molecule and a second molecule are fixed using 3.5% paraformaldehyde (PFA) in phosphate-buffered saline (PBS) for 15 minutes. Cells are washed twice with PBS and permeabilized with 0.25% Triton-X in PBS for 20 minutes. Non-specific epitopes are blocked for one hour using blocking buffer (PBS, 0.1% Triton-X, 10% normal goat serum). Primary immunostaining is performed with the primary antibodies against specific proteins overnight at 4° C. in blocking buffer. Cells are then washed 3× with 0.1% Triton-X in PBS. Secondary immunostaining is performed with the secondary antibodies, for example, AlexaFluor 546 goat anti-rabbit or AlexaFluor 546 goat anti-mouse, in blocking buffer at room temperature for 90 minutes. Cells are washed 3× with 0.1% Triton-X in PBS. DNA is visualized with 2 pg/mL Hoechst dye, staining for 15 minutes in PBS. Finally, Hoechst is removed and replaced with PBS prior to imaging. Controls without primary antibodies are performed to ensure specificity of primary stain.

Microscopy: all images are taken using 60× water immersion objective (NA 1.2) on a Nikon Al laser scanning confocal microscope. An imaging chamber is maintained at 37° C. and 5% C02. For live cell imaging, cells are plated on the fibronectin coated 35-mm glass-bottom dishes and grown typically overnight. For global activation, cells are usually imaged with a 488-nm laser but when the blue light intensity needs to be reduced due to high sensitivity of the optogenetic proteins (e.g., iLID and Cry2), a 440-nm laser is used in conjunction with a dichroic filter for the 488-nm laser. This allows for attenuation of the blue laser intensity at the specimen plane below 0.1 pW. For local activation, a region of interest (ROI) is defined to guide area to be scanned with blue lasers. Fluorescence recovery after photobleaching (FRAP) is performed similarly using ROI.

Image analysis: all data analysis on images is performed using custom-built MATLAB scripts. Briefly, for telomere tracking, raw images are first Gaussian filtered to reduce noise and then peaks corresponding to telomeres are detected based on their peak intensity. Trajectories are generated from a series of detected coordinates based on proximity. To identify and track the boundary of either droplets or heterochromatin, segmented binary images are obtained using the edge detection routine in MATLAB. Analyzed results are manually inspected to check validity.

### SEQUENCES

-GFP11
SEQ ID NO: 1
RDHMVLHEYVNAAGIT
-hnRNPA1
SEQ ID NO: 2
MSKSASPKEPEQLRKLFIGGLSFETTDESLRSHFEQWGTLTDCVVMRDPNTKRSRGFGF
VTYATVEEVDAAMNTTPHKVDGRVVEPKRAVSREDSQRPGAHLTVKKIFVGGIKEDTE
EHHLRDYFEQYGKIEVIEIMTDRGSGKKRGFAFVTFDDHDSVDKIVIQKYHTVKGHNCE
VRKALPKQEMASASSSQRGRRGSGNFGGGRGDGFGGNDNFGRGGNFSGRGGFGGSCG
GGGYGGSGDGYNGFGNDGSNFGGGGSYNDFGNYNNQSSNFGPMKGGNFGGRSSGPYG
GGGQYFAKPQNQGGYGVSSSSSSYGSGRRF
-FUS
SEQ ID NO: 3
MASNDYTQQATQSYGAYPTQPGQGYSQQSSQPYGQQSYSGYSQSTDTSGYGQSSYSSY
GQSQNTGYGTQSTPQGYGSTGGYGSSQSSQSSYGQQSSYPGYGQQPAPSSTSGSYGSSS
QSSSYGQPQSGSYSQQPSYGGQQQSYGQQQSYNPPQGYGQQNQYNSSSGGGGGGGGG
GNYGQDQSSMSSGGGSGGGYGNQDQSGGGGSGGYGQQDRGGRGRGGSGGGGGGGG
GGYNRSSGGYEPRGRGGGRGGRGGMGGSDRGGFNKFGGPRDQGSRHDSEQDNSDNNT
IFVQGLGENVTIESVADYFKQIGIIKTNKKTGQPMINLYTDRETGKLKGEATVSFDDPPSA
KAAIDWFDGKEFSGNPIKVSFATRRADFNRGGGNGRGGRGRGGPMGRGGYGGGGSGG
GGRGGFPSGGGGGGGQQRAGDWKCPNPTCENMNFSWRNECNQCKAPKPDGPGGGPG
GSHMGGNYGDDRRGGRGGYDRGGYRGRGGDRGGFRGGRGGGDRGGFGPGKMDSRG
EHRQDRRERPY
-Ddx4
SEQ ID NO: 4
MGDEDWEAEINPHMSSYVPIFEKDRYSGENGDNFNRTPASSSEMDDGPSRRDHFMKSG
FASGRNFGNRDAGECNKRDNTSTMGGFGVGKSFGNRGFSNSRFEDGDSSGFWRESSND
CEDNPTRNRGFSKRGGYRDGNNSEASGPYRRGGRGSFRGCRGGFGLGSPNNDLDPDEC
MQRTGGLFGSRRPVLSGTGNGDTSQSRSGSGSERGGYKGLNEEVITGSGKNSWKSEAE
GGESSDTQGPKVTYIPPPPPEDEDSIFAHYQTGINFDKYDTILVEVSGHDAPPAILTFEEAN
LCQTLNNNIAKAGYTKLTPVQKYSIPIILAGRDLMACAQTGSGKTAAFLLPILAHMMHD
GITASRFKELQEPECIIVAPTRELVNQIYLEARKFSFGTCVRAVVIYGGTQLGHSIRQIVQG
CNILCATPGRLMDIIGKEKIGLKQIKYLVLDEADRMLDMGFGPEMKKLISCPGMPSKEQ
RQTLMFSATFPEEIQRLAAEFLKSNYLFVAVGQVGGACRDVQQTVLQVGQFSKREKLV
EILRNIGDERTMVFVETKKKADFIATFLCQEKISTTSIHGDREQREREQALGDFRFGKCPV
LVATSVAARGLDIENVQHVINFDLPSTIDEYVHRIGRTGRCGNTGRAISFFDLESDNHLA
QPLVKVLTDAQQDVPAWLEEIAFSTYIPGFSGSTRGNVFASVDTRKGKSTLNTAGFSSSQ
APNPVDDESWD
-hnRNPA1 (isoform b)
SEQ ID NO: 5
MSKSESPKEPEQLRKLFIGGLSFETTDESLRSHFEQWGTLTDCVVMRDPNTKRSRGFGFV
TYATVEEVDAAMNARPHKVDGRVVEPKRAVSREDSQRPGAHLTVKKIFVGGIKEDTEE
HHLRDYFEQYGKIEVIEIMTDRGSGKKRGFAFVTFDDHDSVDKIVIQKYHTVNGHNCEV
RKALSKQEMASASSSQRGRSGSGNFGGGRGGGFGGNDNFGRGGNFSGRGGFGGSRGG
GGYGGSGDGYNGFGNDGGYGGGGPGYSGGSRGYGSGGQGYGNQGSGYGGSGSYDSY
NNGGGGGFGGGSGSNFGGGGSYNDFGNYNNQSSNFGPMKGGNFGGRSSGPYGGGGQY
FAKPRNQGGYGGSSSSSSYGSGRRF

1. A method of identifying an inhibitory activity of an exogeneous agent that disrupts binding of a target peptide to a biomolecule, the method comprising:
(i) contacting a cell with said agent, wherein said cell comprises a target molecule that comprises said target peptide;
(ii) detecting membrane-less bodies comprising said target molecule, wherein said target molecule is incorporated into said membrane-less bodies when said target peptide is released from said binding to said biomolecule and undergoes phase separation at a sub-region of said cell that is separate and distinct from a sub-region where said target peptide binds to said biomolecule;
wherein said detecting comprises measuring a change in an intensity and a size of said membrane-less bodies; and wherein a bigger change in said intensity and said size of said membrane-less bodies relative to a change in an intensity and a size of membrane-less bodies in the absence of said agent indicates that said agent disrupts binding of said target peptide to said biomolecule.

2. The method of claim 1 further comprising:
contacting a second cell with said agent, wherein said second cell comprises a control molecule that comprises a control peptide, wherein said control peptide is a non-target peptide;
detecting comprises measuring a change in an intensity and a size of said first membrane less bodies, and detecting second membrane-less bodies of said second cell comprising said control molecule, wherein said control molecule is incorporated into said second membrane-less bodies when said control peptide undergoes phase separation, and wherein said detecting comprises measuring a change in an intensity and a size of said second membrane-less bodies; and
determining a difference between said change in said intensity and said size of said first-membrane-less bodies and said change in said intensity and said size of said second membrane-less bodies;
wherein a bigger difference between said membrane-less bodies and said second membrane-less bodies relative to a difference between said membrane-less bodies and said second membrane-less bodies in the absence of said agent indicates that said agent disrupts binding of said target peptide to said biomolecule.

3. The method of claim 1, wherein said target peptide is fused to a fluorophore.

4. (canceled)

5. The method of claim 3, wherein said detecting membrane-less bodies is measuring fluorescence, wherein said measuring fluorescence comprises detecting an intensity of fluorescence, quantifying the number of said membrane-less bodies, or a combination thereof.

6. (canceled)

7. The method of claim 1, wherein said target peptide is an intrinsically disordered protein (IDP) or said target peptide comprises an intrinsically disordered region (IDR).

8. The method of claim 1, wherein said membrane-less bodies, first membrane-less bodies, or second membrane-less bodies are nucleoli, Cajal bodies, Stress granule, P-bodies, spliceosomes, or a combination thereof.

9-21. (canceled)

22. The method of claim 1, wherein said target molecule further comprises a full length or truncated low complexity region fused to said target peptide.

23-26. (canceled)

27. The method of claim 7, wherein said IDR is at least a portion of FUS, a portion of Ddx4, a portion of hnRNPA1, a portion of BRD4, a portion of TAF15, a portion of SRSF2 IDR, a portion of SART1, a portion of HSF1, a portion of RNPS1, or a combination thereof.

28. The method of claim 27, wherein said portion of FUS is an N-terminal IDR of FUS (FUSn).

29. The method of claim 3, wherein said fluorophore is mCherry or green fluorescent protein (GFP).

30. (canceled)

31. A method of identifying an inhibitory activity of an exogeneous agent that disrupts binding of a target peptide to a biomolecule, the method comprising:
(i) contacting a cell with said agent, wherein said cell comprises a target molecule that comprises said target peptide, and at least one peptide selected from a light-sensitive receptor and a chemical-sensitive receptor;
(ii) exposing said cell to at least one predetermined wavelength of light, a chemical to which said chemical-sensitive receptor is sensitive, or a combination thereof; and
(iii) detecting membrane-less bodies comprising said target molecule, wherein said target molecule is incorporated into said membrane-less bodies when said target peptide is released from said binding to said biomolecule and undergoes phase separation at a sub-region of said cell that is separate and distinct from a sub-region where said target peptide binds to said biomolecule;
wherein said detecting comprises measuring a change in an intensity and a size of said membrane-less bodies; and wherein a bigger change in said intensity and said size of said membrane-less bodies relative to a change in an intensity and a size of membrane-less bodies in the absence of said agent indicates that said agent disrupts binding of said target peptide to said biomolecule.

32. A method of identifying an inhibitory activity of an exogeneous agent that disrupts binding of a target peptide to a biomolecule, the method comprising:
(i) contacting a cell with said agent, wherein said cell comprises (a) a target molecule that comprises said target peptide and a peptide selected from a group consisting of a light-sensitive receptor, a chemical-sensitive receptor, a light-sensitive oligomerization protein, and a non-light sensitive dimerization module, or a cognate partner thereof; and (b) a second molecule comprising the other of said peptide, or cognate partner thereof, fused to a full length or truncated low complexity protein region or a full length or truncated intrinsically-disordered protein region (IDR);
(ii) exposing said cell to at least one predetermined wavelength of light, a chemical to which said chemical-sensitive receptor is sensitive, or a combination thereof; and
(iii) detecting membrane-less bodies comprising said target molecule, wherein said target molecule and said second molecule are incorporated into said membrane-less bodies when said target peptide is released from said binding to said biomolecule, said peptide of said target molecule interacts with said cognate partner of said second molecule, and said target molecule and said second molecule undergo phase separation at a sub-region of said cell that is separate and distinct from a sub-region where said target peptide binds to said biomolecule,
wherein said detecting comprises measuring a change in an intensity and a size of said membrane-less bodies; and wherein a bigger change in said intensity and said size of said membrane-less bodies relative to a change in an intensity and a size of membrane-less bodies in the absence of said agent indicates that said agent disrupts binding of said target peptide to said biomolecule.

33-34. (canceled)

35. The method of claim 32, wherein said target molecule further comprises a fluorophore fused between said target peptide and said peptide.

36. (canceled)

37. The method of claim 35, wherein said detecting membrane-less bodies is measuring fluorescence, wherein said measuring fluorescence comprises detecting an intensity of fluorescence, quantifying the number of said membrane-less bodies, or a combination thereof.

38. (canceled)

39. The method of claim 32, wherein said membrane-less bodies are nucleoli, Cajal bodies, Stress granule, P-bodies, spliceosomes, or a combination thereof.

40-52. (canceled)

53. The method of claim 32, wherein said IDR is at least a portion of FUS, a portion of Ddx4, a portion of hnRNPA1, a portion of BRD4, a portion of TAF15, a portion of SRSF2 IDR, a portion of SART1, a portion of HSF1, a portion of RNPS1, or a combination thereof.

54. The method of claim 53, wherein said portion of FUS is an N-terminal IDR of FUS (FUSn).

55. The method of claim 32, wherein said light-sensitive receptor is cryptochrome 2 (Cry2), a photolyase homology region of Cry2, phytochrome B (PhyB), PIF, a light oxygen voltage sensing (LOV) domain, Dronpa, or an improved light-induced dimer (iLID).

56. The method of claim 32, wherein said light-sensitive receptor is ssrA and said cognate partner is sspB.

57. The method of claim 32, wherein said fluorophore is mCherry or green fluorescent protein (GFP).

58-101. (canceled)
